(19) United States
(12) Patent Application Publication
(10) Pub. No.: US 2012/0165268 A1
(43) Pub. Date: Jun. 28, 2012

(54) P53 FUSION PROTEINS AND METHODS OF MAKING AND USING THEREOF

(75) Inventors: Xinli Lin, Gaithersburg, MD (US); Michelle Lafevre-Bernt, Novato, CA (US)

(73) Assignee: Proteom Tech, Inc., Costa Mesa, CA (US)

(21) Appl. No.: 13/321,805

(22) PCT Filed: May 28, 2009
(86) PCT No.: PCT/US09/45525

§ 371 Date: Mar. 8, 2012

(51) Int. Cl. A61K 38/17; C07K 14/47; C07K 1/00; A61P 35/00; C07K 1/18; C07K 1/20; C07K 1/22; C07K 1/16; C07K 19/00; C07K 1/14

(52) U.S. Cl. 514/19.3; 530/350; 514/21.2; 530/412; 530/416; 530/417; 530/413

(57) ABSTRACT

Biologically active tetrameric p53 proteins and p53 fusion proteins are provided. These proteins may be generated and refolded into tetrameric form using denatured proteins produced from E. coli. Therapeutic uses of p53 proteins and p53 fusion proteins are also provided.

### FIELD OF THE INVENTION

This invention relates to recombinant tetrameric p53 proteins and p53 fusion proteins and methods for producing the recombinant tetrameric p53 proteins and p53 fusion proteins in bacterial cells.

### BACKGROUND OF THE INVENTION

The p53 tumor suppressor is a transcription factor that resides in the cytosol, and after activation by post-translational modifications it translocates to the nucleus, where it tetramerizes, binds DNA and activates transcription of genes important in cell cycle regulation and DNA repair (Bode, A M and Dong, Z. 2004 Nat Rev Cancer 4:793-805; Braithwaite, A W et al. 2005 Carcinogenesis 26:1161-1169; Coutts, A S and La Thangue, N B 2005, Biochem Biophys Res Commun 331:778-785). Activation of p53 regulates over one hundred cellular genes, and results in cell cycle arrest, giving cells with damaged DNA an opportunity to make repairs, or in the case of irreparable DNA damage, to undergo apoptosis (Lane, D P 1992 Nature 358:15-16; Vousden, K H and Lu X 2002 Nat Rev Cancer 2:594-604). Somatic mutations in the p53 gene are found in many human tumors (Olivier, M et al. 2002 Hum Mutat 19:607-614) and germ line mutations in p53 are responsible for an inherited cancer predisposition, Li-Fraumeni syndrome (Malkin, D 1994 Ann Rev Genet 28:443-465). Recent publications have shown in mouse model that p53 restoration resulted in tumor regression and clearance (Martins, C P et al 2006 Cell 127:1323-34; Ventura, A et al. 2007 Nature 445:661-665; Xue, W et al. 2007 Nature 445:656-660), clearly validated the p53 replacement therapy concept. In addition, the anti-angiogenesis effect of p53 has long been known, and one of the specific effects is the stimulation of extracellular release of collagen-derived peptides such as endostatin and tumstatin, strong inhibitors of angiogenesis (Teodoro, J G, et al 2006 Science 313:968-971).

Despite the fact that p53 is mutated in many human cancers and its role in tumor suppression is well characterized, there are no p53 based anti-neoplastic therapies available. Some of the current standard cancer therapies, including chemotherapy (e.g. cisplatin, carboplatin, oxaliplatin) and radiation therapy, may partly depend on activation of p53 dependent pathways for induction of tumor cell death. However, in many tumors p53 is inactivated by mutation, and these therapies are not as effective as cancer cells with wild type p53. One approach used to treat p53 mutant-expressing tumors has been to use mutant p53 peptides as a vaccine to stimulate the immune system to destroy cells that express mutant variants of p53 (Lomas, M et al 2004 Ann Oncol 15:324-329; Deng, H et al 2002 Cell Immunol 215:20-31). To date, only a modest sensitization of the target tissue to second-line chemotherapy has been elicited from this treatment.

Two strategies have been used to re-activate mutant, inactive p53. The use of small molecules (e.g. nutlin, chalcones and WMC-79) that stabilize or activate p53 function in p53 deficient cancer cells is one promising strategy, but it poses several challenges (Vassilev, L T et al 2005 J Med Chem 48:4491-4499; Kosakowska-Cholody, T et al 2005 Mol Cancer Ther 4:1617-1627). Few molecules have been identified that function to restore the activity of p53 and those that do may be effective for only a small number of p53 mutants. In addition, these molecules may stabilize dominant active mutants of p53, an effect that could be detrimental to patients. C-terminal-derived p53 peptides have also been used to restore wild-type activity to mutant p53 (Snyder, E L et al. 2004 PLoS Biol 2:E36) but this re-activation strategy may also be effective for only a small number of mutants.

For tumors in which p53 cannot be reactivated, or in tumors where p53 expression levels are low, alternative strategies that involve p53 replacement may be developed. Studies in cell culture and animal models have demonstrated a selective sensitivity of p53-deficient tumor cells to the effects of exogenous p53 compared to normal cells (D'Orazi, G et al. 2000 J Gene Med 2:11-21). This suggests that p53 replacement therapy would have fewer side effects than conventional therapies that rely on exploiting active p53-dependent pathways in cells. Gene therapy is one replacement approach that has been used to restore p53 function to cancer cells in cell culture and animal models. In general, however, p53 gene therapy has met with limited success due to the production of adenovirus-neutralizing antibodies and random integration of the p53 gene. Therefore, many of the trials have been discontinued (Zeimet, A G and Marth, C 2003 Lancet Oncol 4:415-422). Protein therapy using p53 may overcome several of the limitations of gene therapy encountered thus far, specifically the safety, toxicity, immune response, and random integration of the transgene. It has been shown that a p53 protein fused with eleven arginine peptide was delivered in oral cancer cell lines and inhibited the proliferation of these cells. Takenobu, et al., Mol. Cancer Ther. 1:1043-1049, 2002. There remains a need to produce biologically active recombinant p53 proteins at a pharmaceutical scale.

All references, publications, and patent applications disclosed herein are hereby incorporated by reference in their entirety.

### BRIEF SUMMARY OF THE INVENTION

The invention provides isolated, refolded p53 proteins in tetrameric form. The invention also provides compositions (including pharmaceutical compositions) comprising the refolded p53 protein in tetrameric form.

The invention also provides isolated, biologically active p53 fusion proteins in tetrameric form. The invention also provides compositions (including pharmaceutical compositions) comprising the biologically active p53 fusion protein in tetrameric form.

In some variations, the p53 fusion protein comprises a p53 protein fused to one or more heterologous peptides. In some variations, the heterologous peptide is fused at the N-terminus and/or the C-terminus of the p53 protein. In some variations, the heterologous peptide comprises the amino acid sequence selected from the group consisting of SEQ ID NO:1, SEQ ID NO:2, SEQ ID NO:3, SEQ ID NO:4, SEQ ID NO:5, and SEQ ID NO:6. In some variations, the heterologous peptide is fused to the p53 protein through a linker peptide. In some variations, the p53 protein comprises the amino acid sequence of SEQ ID NO:7 or amino acid residues 2-392 of SEQ ID NO:7. In some variations, the p53 fusion protein comprises the amino acid sequence selected from the group consisting of SEQ ID NO:8, SEQ ID NO:9, SEQ ID NO:10, SEQ ID NO:11, SEQ ID NO:12, and SEQ ID NO:13. In some variations, the p53 fusion protein comprises the amino acid sequence selected from the group consisting of SEQ ID NO:8, SEQ ID NO:9, SEQ ID NO:10, SEQ ID NO:11, SEQ ID NO:12, and SEQ ID NO:13, wherein the N-terminal amino acid methionine is removed.

In some variations, at least about 70%, at least 75%, at least about 80%, at least about 85%, or at least about 90% of the p53 protein or the p53 fusion protein in the composition is in tetrameric form.

The invention also provides methods of producing a biologically active, refolded p53 protein or p53 fusion protein in tetrameric form comprising refolding the denatured p53 protein or p53 fusion protein (e.g., generated from bacterial cells) into a biologically active tetrameric form.

In some variations, the method comprises: a) solubilizing a denatured p53 protein or p53 fusion protein with a solubilization buffer comprising a high concentration of chaotroph, a reducing agent, and having a pH of about 8.5 to about 12.0, to produce a solubilized p53 protein or p53 fusion protein solution; b) diluting the solubilized p53 protein or p53 fusion protein solution with a refolding buffer by adding the solubilized p53 protein or p53 fusion protein solution into the refolding buffer to produce a diluted solubilized p53 protein or p53 fusion protein solution, wherein the refolding buffer comprises Tris, L-Arginine, a detergent, a divalent cation ion, a chaotroph, or any combination thereof; and c) reducing the pH of the diluted solubilized p53 protein or p53 fusion protein solution to a pH of about 7.5 to about 8.5, wherein said pH reducing step is carried out over a period of at least about 20 hours, thereby producing a refolded, biologically active tetrameric p53 protein or p53 fusion protein. In some variations, the method further comprises purifying the tetrameric p53 protein or p53 fusion protein.

The invention also provides methods for treating cancer in an individual comprising administering to the individual in need thereof an effective amount of the composition comprising a p53 fusion protein in tetrameric form described herein.

The invention also provides a kit comprising the composition comprising a biologically active p53 fusion protein in tetrameric form described herein. In some embodiments, the kit further comprises a package insert indicating that the composition can be used for treating cancer.

It is to be understood that one, some, or all of the properties of the various embodiments and variations described herein may be combined to form other embodiments and variations of the present invention. These and other aspects of the invention will become apparent to one of skill in the art.

### BRIEF DESCRIPTION OF THE DRAWINGS

FIG. 1A shows the amino acid sequence of PTI-p53 (SEQ ID NO:8). The underlined amino acids represent the N-terminal peptide fused to wild-type p53 to facilitate expression in inclusion bodies.

FIG. 1B shows the amino acid sequence of GnRH-p53-v1 (SEQ ID NO:9). The GnRH peptide and polyglycine linker peptide are underlined.

FIG. 2A shows the Sephacryl S-300 chromatogram of refolded PTI-p53. The positions of two peaks are indicated as A and B. Peak A is the misfolded peak, and peak B is the refolded tetrameric p53 peak.

FIG. 2B shows the SDS-PAGE analysis of column fractions shown in FIG. 2A. Monomers, dimers and tetramers of p53 are indicated by the arrows to the right of the gel. Lane 1, MW Std; Lanes 2-5, column fractions from Peak A (misfolded); Lanes 6-13, column fractions from Peak B (tetrameric p53).

FIG. 3A shows the SDS-PAGE analysis of purified p53 tetramer (PTI-p53) incubated with different buffers at 37° C. for 14 h before loaded onto SDS-PAGE. Lane 1: 20 mM Tris, 0.3 M KCl, 10% glycerol, pH 7.8; Lane 2: 8 M urea, 0.1 M Tris, 1 mM Glycine, pH 10 (8 M urea buffer); Lane 3: 8 M urea buffer with 13 mM EDTA; Lane 4: 8 M urea buffer with 0.25% Sacosyl; Lane 5: 8 M urea buffer with 2.5% Tween 20; Lane 6: 8 M urea buffer with 0.25% TMAO; Lane 7: 8 M urea buffer with 0.25% Zwittergent 3-12; and Lane 8: 8 M urea buffer with 2.5% Nonidet P40. The lower bands at about 10 KD shown in Lane 5 and Lane 8 are Tween 20 and Nonidet P40, respectively.

FIG. 3B shows Dynamic Light Scattering analysis of PTI-p53 tetramer at different temperature. Average effective diameter is shown on the y-axis.

FIG. 3C shows Dynamic Light Scattering analysis of misfolded p53 at different temperature. Average effective diameter is shown on the y-axis.

FIG. 3D shows results of DNA binding analysis of PTI-p53 and commercially available p53 (ActiveMotif).

FIG. 3E shows a comparison of DNA binding of PTI-p53 and commercially available p53 at the lowest concentration (right columns) and the highest concentration (left columns) Student t-test value=0.005.

FIG. 4 shows the results of TUNEL analysis of PC3 cells transfected with PTI-p53. Panel A, untransfected, DAPI stained nuclei of the cells; Panel B, untransfected, TUNEL stained; Panel C, p53 transfected, DAPI stained; Panel D, p53 transfected, TUNEL stained; Panel E, TUNEL positive control; Panel F, β-gal stain for transfection efficiency.

FIG. 5A shows the Sephacryl S-300 chromatogram of refolded GnRH-p53 (SEQ ID NO:9). The positions of two peaks are indicated as P1 and P2. Peak P1 is the misfolded peak, and peak P2 is the refolded tetrameric GnRH-p53 peak.

FIG. 5B shows the SDS-PAGE analysis of column fractions shown in FIG. 5A. Lane 1, MW Std; Lanes 2-7, column fractions from Peak P1 (misfolded); Lanes 8-10, column fractions from Peak P2 (tetrameric GnRH-p53).

FIG. 5C shows a comparison of DNA binding of equal amount of GnRH-p53 and commercially available p53 (ActiveMotif). Reactions were carried out in triplicate. OD₄₅₀ is plotted on the y-axis.

FIG. 5D shows results of DNA binding analysis of GnRH-p53 and commercially available p53. Reactions were carried out in triplicate. OD₄₅₀ is plotted on the y-axis.

FIG. 6 shows the results of MTS proliferation assays. Panel A, DU145 cells; Panel B, OVCAR3 cells; and Panel C, MDA-MB-231 cells. Cells were treated with GnRH or GnRH-p53 for 72 hours at 25, 0.925, 0.108, 0.011, or 0 μg/ml as indicated in the legend to the right of the graph and MTS assay was performed. OD₄₉₀ is plotted on y-axis. Panel D, IC50 of each cell line in μg/ml determined by plotting % control growth v. log concentration.

FIG. 7 shows the results of MTS proliferation assays. DU145 cells (top panel), OVCAR3 cells (middle panel) and MDA-MB-231 cells (bottom panel) were treated with GnRH-p53 or PTI-p53 for 72 hours at 100, 22.1, 2.46, 0.82 or 0.24 μg/ml as indicated in the legend to the right of the graph and MTS assay was performed. OD₄₉₀ is plotted on y-axis.

FIG. 8A shows the results of the nucleosome formation ELISA. DU145, OVCAR3 and MDA-MB-231 cells were treated with GnRH-p53, PTI-p53, buffer alone or untreated. OD₄₅₀ is plotted on y-axis. Arrows indicate GnRH-p53 induced nucleosome formation in DU145 cells and OVCAR3 cells.

FIG. 8B shows the results of TUNEL analysis of OVCAR3 cells treated with GnRH-p53, PTI-p53 or buffer alone or untreated, as indicated beneath the panels.

FIG. 9A shows p53 internalization and translocation. OVCAR3 cells treated with GnRH-p53 for the indicated times and stained with BP52-12 anti-p53 monoclonal antibody and Alexa 555 secondary antibody (top panels) or with TOTO3 nuclear stain (bottom panels).

FIG. 9B shows confocal microscopy of cells treated with GnRH-p53 (top two rows of panels) or PTI-p53 (bottom panels) for the indicated times.

FIG. 9C shows colocalization statistics for confocal Z-stacks for GnRH-p53 and nuclear localization. Data represents the percent of p53 above threshold that localizes with nuclear staining at each time point.

FIG. 10 shows the amino acid sequence of GNRH-p53-v2 (SEQ ID NO:10) and HA-GNRH-p53-v2 (SEQ ID NO:11). GNRH-p53-v2 contains, from N-terminus to C-terminus, amino acid methionine, a GnRH sequence (SSQHWSYGLRPG (SEQ ID NO:2)), a linker (GGGS (SEQ ID NO:21)), and wild type p53 sequence without N-terminus methionine. HA-GNRH-p53-v2 contains, from N-terminus to C-terminus, amino acid methionine, a HA sequence (GLFGAIAGFIENGWEGMID (SEQ ID NO:5), a linker (GGGGGG (SEQ ID NO:22)), a GnRH sequence (SSQHWSYGLRPG (SEQ ID NO:2)), a linker (GGGGS (SEQ ID NO:23)), and wild type p53 sequence without N-terminus methionine.

### DETAILED DESCRIPTION OF THE INVENTION

The instant invention provides isolated p53 proteins and p53 fusion proteins in tetrameric form, compositions comprising the isolated p53 proteins or p53 fusion proteins, methods for producing the p53 proteins or p53 fusion proteins in tetrameric form from inclusion bodies of bacterial cells, and methods of using the p53 fusion proteins for treating cancer in a subject.

The practice of the present invention will employ, unless otherwise indicated, conventional techniques of immunology, molecular biology, microbiology, cell biology and recombinant DNA, which are within the skill of the art. See, e.g., Molecular Cloning: a laboratory manual, 3^{rd} edition Sambrook, et al. (2001); Current Protocols In Molecular Biology F. M. Ausubel, et al. eds., (1987); the series Methods In Enzymology, Academic Press, Inc.; PCR 2: A Practical Approach, M. J. MacPherson, B. D. Hames and G. R. Taylor, eds. (1995), and Antibodies, A Laboratory Manual, Harlow and Lane, eds. (1988).

### A. Definitions

As used herein, “treatment” or “treating” is an approach for obtaining beneficial or desired results including and preferably clinical results. For purposes of this invention, beneficial or desired clinical results include, but are not limited to, one or more of the following: reducing the growth of (or destroying) cancerous cells, reducing metastasis of cancerous cells found in cancers, shrinking the size of the tumor, decreasing symptoms resulting from the disease, increasing the quality of life of those suffering from the disease, decreasing the dose of other medications required to treat the disease, delaying the progression of the disease, and/or prolonging survival of individuals.

An “effective amount” of a drug, or pharmaceutical composition is an amount sufficient to effect beneficial or desired results including clinical results such as shrinking the size of the tumor (in the cancer context), retardation of cancerous cell growth, decreasing one or more symptoms resulting from the disease, increasing the quality of life of those suffering from the disease, decreasing the dose of other medications required to treat the disease, enhancing effect of another medication such as via targeting, delaying the progression of the disease, and/or prolonging survival of individuals. An effective amount can be administered in one or more administrations. For purposes of this invention, an effective amount of drug, compound, or pharmaceutical composition is an amount sufficient to reduce the proliferation of (or destroy) cancerous cells and to reduce and/or delay the development, or growth, of metastases of cancerous cells, either directly or indirectly. As is understood in the cancer clinical context, an effective amount of a drug, compound, or pharmaceutical composition may or may not be achieved in conjunction with another drug, compound, or pharmaceutical composition. Thus, an “effective amount” may be considered in the context of administering one or more therapeutic agents, and a single agent may be considered to be given in an effective amount if, in conjunction with one or more other agents, a desirable result may be or is achieved.

As used herein, “delaying development of metastasis” means to defer, hinder, slow, retard, stabilize, and/or postpone development of metastasis. This delay can be of varying lengths of time, depending on the history of the cancer and/or individual being treated. As is evident to one skilled in the art, a sufficient or significant delay can, in effect, encompass prevention, in that the individual does not develop the metastasis.

As used herein, “in conjunction with” refers to administration of one treatment modality in addition to another treatment modality, such as administration of a p53 fusion protein and other anti-cancer drug. As such, “in conjunction with” refers to administration of one treatment modality before, during or after administration of the other treatment modality to the individual.

A “subject” or an “individual” is a mammal, more preferably a human. Mammals include, but are not limited to, farm animals, sport animals, pets (such as cats, dogs, horses), primates, mice and rats.

“Heterologous” means derived from a genotypically distinct entity from that of the rest of the entity to which it is compared. For example, a polypeptide from a non-p53 protein is heterologous to the p53 protein.

It should be noted that, as used herein, the singular form “a”, “an”, and “the” includes plural references unless indicated otherwise.

When “about” is used to describe a range, the term applies to both lower and upper value of a range. For example, “about X to Y” means “about X to about Y”. Reference to “about” a value or parameter herein includes (and describes) embodiments that are directed to that value or parameter per se. For example, description referring to “about X” includes description of “X.”

As used herein, “isolated” refers to a molecule (such as a protein or a nucleic acid) which has been identified and separated and/or recovered from a component of its natural environment.

It is understood that aspect and embodiments of the invention described herein include “consisting” and/or “consisting essentially of” aspects and embodiments.

### B. p53 Proteins and Fusion Proteins

The invention provides isolated tetrameric form of p53 proteins and p53 fusion proteins. The invention also provides compositions comprising the tetrameric form of p53 proteins or p53 fusion proteins.

In some variations, the p53 protein is a human p53 protein. In some variations, the p53 protein is a naturally occurring variant or a naturally occurring mutant. The wild type human p53 sequence is shown below. In some variations, the p53 protein comprises the amino acid sequence of SEQ ID NO:7. In some variations, the p53 protein comprises the amino acid sequence of SEQ ID NO:7, wherein one, two, three, four or five amino acid residues in the N-terminus and/or one, two, three, four, or five amino acid residues in the C-terminus of SEQ ID NO:7 are deleted. In some variations, the p53 protein comprises the amino acid sequence selected from the group consisting of residues 2-393 of SEQ ID NO:7, residues 3-393 of SEQ ID NO:7, residues 4-393 of SEQ ID NO:7, residues 5-393 of SEQ ID NO:7, residues 6-393 of SEQ ID NO:7, residues 2-392 of SEQ ID NO:7, residues 2-391 of SEQ ID NO:7, residues 2-390 of SEQ ID NO:7, residues 2-389 of SEQ ID NO:7, and residues 2-388 of SEQ ID NO:7.

```
Wild-type human p53
(SEQ ID NO: 7)
                10 20 30 40 50 60
MEEPQSDPSV EPPLSQETFS DLWKLLPENN VLSPLPSQAM DDLMLSPDDI EQWFTEDPGP
                70 80 90 100 110 120
DEAPRMPEAA PPVAPAPAAP TPAAPAPAPS WPLSSSVPSQ KTYQGSYGFR LGFLHSGTAK
              130 140 150 160 170 180
SVTCTYSPAL NKMFCQLAKT CPVQLWVDST PPPGTRVRAM AIYKQSQHMT EVVRRCPHHE
              190 200 210 220 230 240
RCSDSDGLAP PQHLIRVEGN LRVEYLDDRN TFRHSVVVPY EPPEVGSDCT TIHYNYMCNS
              250 260 270 280 290 300
SCMGGMNRRP ILTIITLEDS SGNLLGRNSF EVRVCACPGR DRRTEEENLR KKGEPHHELP
              310 320 330 340 350 360
PGSTKRALPN NTSSSPQPKK KPLDGEYFTL QIRGRERFEM FRELNEALEL KDAQAGKEPG
              370 380 390
GSRAHSSHLK SKKGQSTSRH KKLMFKTEGP DSD
```

It has been shown that PRIMA-1 reactivates mutant p53 by covalently binding to the core domain and the covalently modified mutant p53 induced apoptosis in tumor cells. Lamber et al., Cancer Cell 15:376-88, 2009. The refolded tetrameric p53 proteins and variants described herein may be used in screening small molecules that activate mutant p53 for the development of anticancer drugs. The refolded tetrameric p53 proteins and variants may be used in co-crystallization with binding molecules to study the mechanism of the activation for designing more efficient anticancer drugs.

In some variations, the p53 fusion protein comprises a p53 protein fused to one or more heterologous peptides. In some variations, the p53 protein is fused to a peptide that targets a broad spectra of cancer cell types and internalizes the fusion protein into the cancer cell. In some variations, the p53 protein is fused to a peptide that targets a specific cancer cell and internalizes the fusion protein into the cancer cell. In some variations, the p53 protein is further fused to a peptide that allows efficient endosomal escape and nuclear localization of the fusion protein. In some variations, one, two, or more heterologous peptides are fused to the p53 protein. The heterologous peptides may be fused to the N-terminus and/or the C-terminus of the p53 protein.

The fusion proteins of the invention may be characterized by one or more of the following characteristics: (a) ability to be internalized by a cancer cell; (b) ability to enter into nucleus of a cancer cell; and (c) ability to inhibit proliferation and induce apoptosis of a cancer cell.

In some variations, a ganadotropin-releasing hormone sequence such as GnRH-v1: HWSYGLRPG (SEQ ID NO:1) and GnRH-v2: SSQHWSYGLRPG (SEQ ID NO:2) may be fused to the p53 protein. In some variations, R11 sequence RRRRRRRRRRR (SEQ ID NO:3) may be fused to the p53 protein. In some variations, TAT sequence GRKKRRQRRRPP (SEQ ID NO:4) may be fused to the p53 protein. In some variations, an N-terminal sequence of influenza virus hemagglutinin protein (HA2) (such as the sequence GLFGAIAGFIENGWEGMID (SEQ ID NO:5)) may be used as the peptide for efficient endosomal escape and nuclear localization of the fusion protein. In some variations, peptide HA-GnRH-v2 GLFGAIAGFIENGWEGMIDGGGGGGSSQHWSYGLRPG (SEQ ID NO:6) may be fused to the p53 protein.

A heterologous peptide may be fused to the p53 protein through a linker sequence. A linker sequence may be used between various sequences (such as between the internalization peptide and the p53 protein, and/or between the internalization peptide and the peptide that allows efficient endosomal escape and nuclear localization). In some variations, the linker sequence comprises 3 to 6 glycine residues. For example, the following linker sequences may be used: PGGGGS (SEQ ID NO:17), ESGGGGSPG (SEQ ID NO:18), SPGGGGSPG (SEQ ID NO:19), APGAGAGPG (SEQ ID NO:20), GGGS (SEQ ID NO:21), GGGGGG (SEQ ID NO:22), GGGGS (SEQ ID NO:23).

In some variations, the fusion protein comprises the amino acid sequence selected from the group consisting of SEQ ID NOS:8-13. In some variations, one, two, three, four, or five amino acid residues at the N-terminus and/or one, two, three, four, or five amino acid residues of the fusion protein are removed. In some variations, the first methionine at the N-terminus of the fusion protein is removed.

```
p53-R11
(SEQ ID NO: 12)
                10 20 30 40 50 60
MEEPQSDPSV EPPLSQETFS DLWKLLPENN VLSPLPSQAM DDLMLSPDDI EQWFTEDPGP
                70 80 90 100 110 120
DEAPRMPEAA PRVAPAPAAP TPAAPAPAPS WPLSSSVPSQ KTYQGSYGFR LGFLHSGTAK
              130 140 150 160 170 180
SVTCTYSPAL NKMFCQLAKT CPVQLWVDST PPPGTRVRAM AIYKQSQHMT EVVRRCPHHE
              190 200 210 220 230 240
RCSDSDGLAP PQHLIRVEGN LRVEYLDDRN TFRHSVVVPY EPPEVGSDCT TIHYNYMCNS
              250 260 270 280 290 300
SCMGGMNRRP ILTIITLEDS SGNLLGRNSF EVRVCACPGR DRRTEEENLR KKGEPHHELP
              310 320 330 340 350 360
PGSTKRALPN NTSSSPQPKK KPLDGEYFTL QIRGRERFEM FRELNEALEL KDAQAGKEPG
              370 380 390 400 410
GSRAHSSHLK SKKGQSTSRH KKLMFKTEGP DSDPGGGGSR RRRRRRRRRR
p53-TAT
(SEQ ID NO: 13)
                10 20 30 40 50 60
MEEPQSDPSV EPPLSQETFS DLWKLLPENN VLSPLPSQAM DDLMLSPDDI EQWFTEDPGP
                70 80 90 100 110 120
DEAPRMPEAA PRVAPAPAAP TPAAPAPAPS WPLSSSVPSQ KTYQGSYGFR LGFLHSGTAK
              130 140 150 160 170 180
SVTCTYSPAL NKMFCQLAKT CPVQLWVDST PPPGTRVRAM AIYKQSQHMT EVVRRCPHHE
              190 200 210 220 230 240
RCSDSDGLAP PQHLIRVEGN LRVEYLDDRN TFRHSVVVPY EPPEVGSDCT TIHYNYMCNS
              250 260 270 280 290 300
SCMGGMNRRP ILTIITLEDS SGNLLGRNSF EVRVCACPGR DRRTEEENLR KKGEPHHELP
              310 320 330 340 350 360
PGSTKRALPN NTSSSPQPKK KPLDGEYFTL QIRGRERFEM FRELNEALEL KDAQAGKEPG
              370 380 390 400 410
GSRAHSSHLK SKKGQSTSRH KKLMFKTEGP DSDPGGGGSG RKKRRQRRRP P
```

The invention also provides variants of any of the p53 proteins and p53 fusion proteins described herein. Variants may include one, two, three, or more amino acid substitutions, deletions or additions. Variants may be from natural mutations or human manipulation. Changes can be of a minor nature, such as conservative amino acid substitutions that do not significantly affect the folding or activity of the protein. Recombinant DNA technology known to those skilled in the art can be used to create novel mutant proteins or mutants including single or multiple amino acid substitutions, deletions, or additions. Such modified polypeptides can show, e.g., enhanced activity, increased stability, or decreased activity. In addition, they may be purified in higher yields and show better solubility than the corresponding natural polypeptide, at least under certain purification and storage conditions. Variants also include those having one or more amino acid residues deleted, added, or substituted to generate polypeptides that are better suited for expression, scale up, etc., in the host cells chosen. In some embodiments, amino acid sequences of the variants are at least about any of 70%, 75%, 80%, 85%, 90%, 95%, 96%, 97%, 98%, or 99% identical to a p53 protein or p53 fusion protein described herein.

Two polypeptide sequences are said to be “identical” if the sequence of amino acids in the two sequences is the same when aligned for maximum correspondence as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, 40 to about 50, in which a sequence may be compared to a reference sequence of the same number of contiguous positions after the two sequences are optimally aligned.

Optimal alignment of sequences for comparison may be conducted using the Megalign program in the Lasergene suite of bioinformatics software (DNASTAR, Inc., Madison, Wis.), using default parameters. This program embodies several alignment schemes described in the following references: Dayhoff, M. O. (1978) A model of evolutionary change in proteins—Matrices for detecting distant relationships. In Dayhoff, M. O. (ed.) Atlas of Protein Sequence and Structure, National Biomedical Research Foundation, Washington D.C. Vol. 5, Suppl. 3, pp. 345-358; Hein J., 1990, Unified Approach to Alignment and Phylogenes pp. 626-645 Methods in Enzymology vol. 183, Academic Press, Inc., San Diego, Calif.; Higgins, D. G. and Sharp, P. M., 1989, CABIOS 5:151-153; Myers, E. W. and Muller W., 1988, CABIOS 4:11-17; Robinson, E. D., 1971, Comb. Theor. 11:105; Santou, N., Nes, M., 1987, Mol. Biol. Evol. 4:406-425; Sneath, P. H. A. and Sokal, R. R., 1973, Numerical Taxonomy the Principles and Practice of Numerical Taxonomy, Freeman Press, San Francisco, Calif.; Wilbur, W. J. and Lipman, D. J., 1983, Proc. Natl. Acad. Sci. USA 80:726-730.

Preferably, the “percentage of sequence identity” is determined by comparing two optimally aligned sequences over a window of comparison of at least 20 positions, wherein the portion of the polypeptide sequence in the comparison window may comprise additions or deletions (i.e. gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical amino acid residue occurs in both sequences to yield the number of matched positions, dividing the number of matched positions by the total number of positions in the reference sequence (i.e. the window size) and multiplying the results by 100 to yield the percentage of sequence identity.

Variants also include conjugate comprising any of the fusion protein embodiments described herein, e.g., conjugated or fused to a half life extending moiety, such as a PEG or a peptide.

Variants also include functional equivalent variants. Functional equivalent variants are identified by any one or more of the following criteria: (a) ability to be internalized by a cancer cell; (b) ability to enter into nucleus of a cancer cell; and (c) ability to inhibit proliferation and induce apoptosis of a cancer cell. The biological activities of the variants may be tested using methods known in the art and methods described herein. In some variations, functional equivalent variants have at least about any of 50%, 60%, 70%, 75%, 80%, 85%, 90%, or 95% of activity as compared to wildtype p53 protein or the fusion protein having wild type p53 protein with respect to one or more of the biological assays described above (or known in the art).

### C. Methods of Refolding and Purification of Tetrameric p53 Proteins and p53 Fusion Proteins

The invention also provides methods for producing p53 proteins or p53 fusion proteins from inclusion bodies containing the p53 proteins or the p53 fusion proteins from bacterial cells (e.g. E. coli) which have been engineered to produce the proteins. The methods described herein may also be used to produce any variants described herein.

Methods for refolding proteins from E. coli inclusion bodies have been reported. See, e.g., U.S. Pat. No. 6,583,268; U.S. Pub. Nos. US 2003/070242; US 2003/0199676; US 2004/0265298; and US 2005/0227920; and PCT Pub. Nos. WO 03/039491; WO 2004/094344; WO 01/55174; and WO 2005/05830.

Recombinant bacterial host cells (e.g. E. coli) may be engineered to produce any of the proteins (including the p53 proteins, p53 fusion proteins, and variants) using any convenient technology. Most commonly, a DNA sequence encoding the desired protein is inserted into the appropriate site in a plasmid-based expression vector which provides appropriate transcriptional and translational control sequences, although expression vectors based on bacteriophage genomic DNA are also useful. It is generally preferred that the transcriptional control sequences are inducible by a change in the environment surrounding the host cells (such as addition of a substrate or pseudosubstrate to which the transcriptional control sequences are responsive), although constitutive transcriptional control sequences are also useful. As is standard in the art, it is also preferred that the expression vector include a positive selectable marker (e.g., the β-lactamase gene, which confers resistance to ampicillin) to allow for selection against bacterial host cells which do not contain the expression vector. The invention provides polynucleotides and vectors comprising a nucleotide sequence encoding any of the p53 proteins and p53 fusion proteins described herein.

The bacterial host cells are typically cultured in a liquid growth medium for production of the p53 protein or p53 fusion protein under conditions appropriate to the host cells and expression vector. Preferably, the host cells are cultured in a bacterial fermenter to maximize production, but any convenient method of culture is acceptable (e.g., shaken flask, especially for cultures of less than a liter in volume). As will be apparent to those of skill in the art, the exact growing conditions, timing and rate of media supplementation, and addition of inducing agent (where appropriate) will vary according to the identity of the host cells and the expression construct.

After the bacterial host cells are cultured to the desired density (and after any necessary induction of expression), the cells are collected. Collection is typically conveniently effected by centrifugation of the growth medium, although any other convenient technique may be used. The collected bacterial host cells may be washed at this stage to remove traces of the growth medium, most typically by resuspension in a simple buffer followed by centrifugation (or other convenient cell collection method). At this point, the collected bacterial host cells (the “cell paste”) may be immediately processed in accordance with the invention, or it may be frozen for processing at a later time.

The cells of the cell paste are lysed to release the protein-containing inclusion bodies. Preferably, the cells are lysed under conditions in which the cellular debris is sufficiently disrupted that it fails to appear in the pellet under low speed centrifugation. Commonly, the cells are suspended in a buffer at about pH 5 to 9, preferably about 6 to 8, using an ionic strength of the order of about 0.01 M to 2 M preferably about 0.1-0.2 M (it is apparently undesirable to use essentially zero ionic strength). Any suitable salt, including NaCl can be used to maintain an appropriate ionic strength level. The cells, while suspended in the foregoing buffer, are then lysed by techniques commonly employed such as, for example, mechanical methods such as freeze/thaw cycling, the use of a Manton-Gaulin press, a French press, or a sonic oscillator, or by chemical or enzymatic methods such as treatment with lysozyme. It is generally desirable to perform cell lysis, and optionally bacterial cell collection, under conditions of reduced temperature (i.e., less than about 20° C.).

Inclusion bodies are collected from the lysed cell paste using any convenient technique (e.g., centrifugation), then washed. If desired, the collected inclusion bodies may be washed. Inclusion bodies are typically washed by resuspending the inclusion bodies in a wash buffer, typically the lysis buffer, preferably with a detergent added (e.g., 1% TRITON X-100®), then recollecting the inclusion bodies. The washed inclusion bodies are then dissolved in solubilization buffer. Solubilization buffer comprises a high concentration of a chaotroph, a pH buffer that buffers the solution to a high pH, and one or more reducing reagents. The solubilization buffer may optionally contain additional agents, such as redox reagents, and scavengers to neutralize protein-damaging free-radicals.

The instant invention utilizes urea as an exemplary chaotroph in the solubilization buffer, although guanidine hydrochloride (guanidine HCl) may also be used. Useful concentrations of urea in the solubilization buffer include about 7.5 M to about 9 M, about 8 M to about 8.5M, or about 8 M. When the chaotroph is guanidine HCl, useful concentrations include about 5 M to about 7 M, or about 5.5 M to about 6.5 M, or about 6 M.

The pH of the solubilization buffer is high, viz., in excess of pH 8.0, for example pH 9.0. Useful pH levels in the solubilization buffer are in the range of about 8.0 to about 12.0, about 9.0 to about 12.0, about 9.0 to about 11.0, about 9.5 to about 10.5, about 10.0 to about 10.5, about 10, about 10.5, about 10.8, about 11.0, about 11.5, or about 12.0. As will be apparent to those of skill in the art, any pH buffering agent (or combination of agents) which effectively buffer at high pH are useful, although pH buffers which can buffer in the range of about pH 8 to about pH 9 or 10 are particularly useful. Useful pH buffering agents include tris (tris(hydroxymethyl)aminomethane), bicine (N,N-Bis(2-hydroxyethyl)glycine), HEPBS (2-Hydroxy-1,1-bis[hydroxymethyl]ethyl)amino-1-1-propanesulfonic acid), TAPS ([(2-Hydroxy-1,1-bis[hydroxymethyl]ethyl)amino]-1-propanesulfonic acid), AMPD (2-Amino-2-methyl-1,3-propanediol), HEPBS (N-(2-Hydroxyethyl)piperazine-N′-(4-butanesulfonic acid)), and the like. The pH buffering agent is added to a concentration that provides effective pH buffering, such as from about 50 to about 150 mM, about 75 mM to about 125 mM, or about 100 mM.

Reducing reagents are included in the solubilization buffer to reduce disulfide bonds and maintain cysteine residues in their reduced form. Useful reducing reagents include beta-mercaptoethanol, dithiothreitol, and the like. Additionally, the solubilization buffer may contain disulfide reshuffling or “redox” reagents (e.g., a combination of oxidized and reduced glutathione). When the redox reagents are oxidized and reduced glutathione (GSSG and GSH, respectively), the inventor has found that useful concentrations include about 0.1 mM to about 11 mM and useful ratios include about 10:1, about 5:1, and about 1:1 (GSH:GSSG).

The solubilization buffer may contain additional components. For example, a free-radical scavenger may be added to reduce or eliminate free-radical-mediated protein damage, particularly if urea is used as the chaotroph and it is expected that a urea-containing protein solution will be stored for any significant period of time. Suitable free-radical scavengers include glycine (e.g., at about 0.5 to about 2 mM, or about 1 mM) and other amino acids and amines.

An exemplary solubilization buffer comprises about 8 M urea, about 0.1 M Tris, about 1 mM glycine, about 10 mM beta-mercaptoethanol, about 10 mM dithiothreitol (DTT), and about 1 mM reduced glutathion (GSH), pH about 10.5.

The inclusion body/solubilization buffer mixture is incubated to allow full solubilization. The incubation period is generally from about six hours to about 24 hours, and more commonly about eight to about 14 hours or about 12 hours. The inclusion body/solubilization buffer mixture incubation may be carried out at reduced temperature, commonly at about 4° C. to about 10° C.

After the incubation is complete, the inclusion body/solubilization buffer mixture is clarified to remove insoluble debris. Clarification of the mixture may be accomplished by any convenient means, such as filtration (e.g., by use of depth filtration media) or by centrifugation. Clarification should be carried out at reduced temperature, such as at about 4° C. to about 10° C.

The clarified mixture is then diluted using the same solubilization buffer to achieve the appropriate protein concentration for refolding. Protein concentration may be determined using any convenient technique, such as Bradford assay, light absorption at 280 nm (A₂₈₀), and the like. The inventor has found that a solution having an A₂₈₀ of about 2.0 to about 10.0 (e.g., about any of 2.0, 3.0, 4.0, 5.0, 6.0, 7.0, 8.0, 9.0, and 10.0), is appropriate for use in the instant methods. For example, protein concentration at a final concentration of 2 mg/ml is appropriate. If desired, this mixture may be held, refrigerated (e.g. at 4° C.), for later processing, although the mixture is not normally held for more than about four weeks.

The concentration-adjusted inclusion body solution is first rapidly diluted with refolding buffer. The dilution is performed by adding inclusion body solution into the refolding buffer. The inclusion body solution may be diluted about 10 to about 100 fold, about 10 to about 50 fold, about 10 to about 25 fold, about 15 to about 25 fold, or about 20 fold with refolding buffer. The inclusion body solution is diluted to reduce urea and protein concentration. The final protein concentration after dilution may be about 0.01 mg/ml to about 1 mg/ml, about 0.1 mg/ml to about 0.5 mg/ml. The refolding buffer contains one or more detergents and a pH buffer. The refolding buffer may also contain a low concentration of chaotroph, a disulfide reshuffling reagent, and a divalent cation ion. The refolding buffer may include additional agents, such as free-radical scavengers. “Rapid” dilution, within the context of the invention means over a period of less than about 25 minutes, and the dilution process is generally carried out during periods of about two minutes to about 25 minutes, or about five to about 20 minutes. The diluted solubilized p53 or p53 fusion protein solution is typically held for one to two hours following the completion of the rapid dilution process. One or more detergents may be included in the refolding buffer.

The pH of the refolding buffer may be the same as the solubilization buffer. The pH buffering agent in the refolding buffer may be any buffering agent or combination of buffering agents that are effective pH buffers at pH levels of about 8 to about 9 or about 10 or about 10.5. Useful pH buffering agents include tris (tris(hydroxymethyl)aminomethane), bicine (N,N-Bis(2-hydroxyethyl)glycine), HEPBS (2-Hydroxy-1,1-bis[hydroxymethyl]ethyl)amino]-1-propanesulfonic acid), TAPS ([(2-Hydroxy-1,1-bis[hydroxymethyl]ethyl)amino]-1-propanesulfonic acid), and AMPD (2-Amino-2-methyl-1,3-propanediol), HEPBS (N-(2-Hydroxyethyl)piperazine-N′-(4-butanesulfonic acid)). The pH buffering agent is added to a concentration that provides effective pH buffering, such as from about 10 to about 150 mM, about 50 to about 150 mM, about 75 mM to about 125 mM, or about 100 mM.

Additional components useful in the refolding buffer include free-radical scavengers. A free-radical scavenger may be added to reduce or eliminate free-radical-mediated protein damage, particularly if urea is used as the chaotroph and it is expected that a urea-containing protein solution will be stored for any significant period of time. Suitable free-radical scavengers include glycine (e.g., at about 0.5 to about 2 mM, or about 1 mM).

The refolding buffer may further comprise one or more detergent, such as Tween 20, Tween 80, N-lauroylsarcosine, and sodium dodecyl sulfate. The refolding buffer may comprise about 0.01 to about 5% detergent.

An exemplary refolding buffer comprises about 20 mM Tris, about 0.2 M L-arginine, about 0.1 mM ZnCl₂, pH about 10.5.

The pH of the refolding solution is then slowly reduced from elevated pH to near neutral pH using an appropriate acid. In some variations, the pH is reduced to about 7.5 to about 8.5 (for example, to pH about 8.0). The time period for pH reduction can range from about 20-24 hours to about 20 days, about 20 to about 50 hours, about 20 to about 40 hours, about 20 to about 30 hours, about 24 to about 40 hours. The time period for pH reduction can be at least about 20 hours, at least about 24 hours, at least about 30 hours, at least about 40 hours, at least about 48 hours, at least about 50 hours, at least about 4 days, at least about 5 days, at least about 6 days, at least about 8 days, at least about 10 days, at least about 15 days, or about 20 days. In some embodiments, the time period for pH reduction is about 2-5 days. Appropriate acids for pH adjustment will depend on the pH buffer used in the refolding buffer. For example, when the pH buffering agent is tris, the pH should be adjusted with hydrochloric acid (HCl).

Following completion of pH adjustment, the refolding reaction is incubated for a period of about one to two hours to about 18 to 24 hours. The refolding reaction may be carried out at room temperature (e.g., about 18-20° C.) or at slightly reduced temperatures (e.g., about 14-16° C.), depending on the preferences of the practitioner and the available facilities.

In some variations, the p53 protein or p53 fusion protein is refolded into tetrameric form by the following steps: a) solubilizing a denatured p53 protein or p53 fusion protein with a solubilization buffer comprising about 8M urea, about 0.1 M Tris, about 1 mM glycine, about 10 mM beta-mercaptoethanol, about 10 mM DTT, about 1 mM reduced glutathione, pH about 10.5, to produce a solubilized p53 protein or p53 fusion protein solution; b) diluting the solubilized p53 protein or p53 fusion protein solution with a refolding buffer by adding the solubilized p53 protein or p53 fusion protein solution into about 20 volumes of the refolding buffer comprising about 20 mM Tris, about 0.2 M arginine, about 0.1 mM ZnCl₂; and c) reducing the pH of the diluted solubilized p53 protein or p53 fusion protein solution to a pH of about 8.0 over a period of at least about 20 hours, thereby producing the refolded p53 protein or p53 fusion protein in tetrameric form.

Following the refolding reaction, properly refolded p53 protein or p53 fusion protein may be concentrated and further purified. Concentration of the refolded protein may be accomplished using any convenient technique, such as ultrafiltration, diafilitration, chromatography (e.g., ion-exchange, hydrophobic interaction, affinity chromatography, size exclusion chromatography) and the like. Any combination of these techniques may be used. Where practical, it is preferred that concentration be carried out at reduced temperature (e.g., about 4-10° C.). The concentration step may also include a buffer exchange process to remove the detergent in the refolding buffer before purifying the protein.

While any convenient protein purification protocol may be used. In some variations, tangential flow filtration is used to concentrate the refolded proteins and size exclusion chromatography (SEC) is used to purify the refolded proteins.

In some variations, the refolded p53 protein or p53 fusion protein is purified from contaminants. For example, p53 fusion protein may be purified from contaminants which would interfere with therapeutic uses for the p53 fusion protein. The contaminants may include enzymes, hormones, and other proteinaceous or nonproteinaceous solutes from the bacterial cells and cell culture. In some variations, the refolded p53 protein or p53 fusion protein is purified to at least any of about 70%, about 75%, about 80%, about 85%, about 90%, about 95%, and about 99% by weight. Purity of the protein composition can be determined by methods known in the art, such as the Lowry method, amino acid sequencing, and SDS-PAGE under reducing or nonreducing condition using Coomassie blue or silver staining. In some variations, greater than any of about 70%, about 75%, about 80%, about 85%, about 90%, about 95%, about 99% of the p53 protein or p53 fusion protein in the composition is in tetrameric form. The percentage of the p53 protein or p53 fusion protein in tetrameric form may be measured by methods known in the art, such as SDA-PAGE and dynamic light scattering (DLS). See Example 1.

Biological activity of the refolded p53 proteins, p53 fusion proteins, and variants may be measured using any acceptable assay method known in the art. For example, in vitro DNA binding assay, cell proliferation assay, and cell apoptosis assay may be used. See Example 1.

As is well understood in the art, all concentrations and pH values need not be exact and reference to a given value reflects standard usage in the art, does not mean that the value cannot vary.

### D. Compositions, Therapeutic Use, and Kits

The invention also provides compositions (including pharmaceutical compositions) comprising biologically active, tetrameric form of p53 protein, p53 fusion proteins, or variants described herein. The composition may further comprise a pharmaceutical acceptable excipient. The p53 protein, p53 fusion proteins or variants may be in the form of lyophilized formulations or aqueous solutions. Acceptable excipients are nontoxic to recipients at the dosages and concentrations employed, and may comprise buffers such as phosphate, citrate; salts such as sodium chloride; sugars such as sucrose; and/or polyethylene glycol (PEG). See Remington: The Science and Practice of Pharmacy 20th Ed. (2000) Lippincott Williams and Wilkins, Ed. K. E. Hoover. The p53 fusion proteins may be formulated for different routes of drug delivery formulations, such as liquid or lyophilized formulation for intravenous (IV) injection, and dry powered formulation or aerosolization formulation for deep lung delivery. These formulations are known in the art. See, e.g., Drug Delivery to the Lung, Bisgaard H., O'Callaghan C and Smaldone G C, editors, New York; Marcel Dekker, 2002.

In some variations, at least about 70% of the p53 protein or p53 fusion protein in the composition is in tetrameric form. In some variations, at least about 75%, at least about 80%, at least about 85%, at least about 90%, at least about 95%, at least about 99% of the p53 protein or p53 fusion protein in the composition is in tetrameric form. In some variations, the monomers of the tetrameric p53 protein or p53 fusion protein in the composition are not chemically cross-linked with each other.

The invention also provides methods for treating cancer in a subject having cancer comprising administering to the subject an effective amount of a p53 protein or p53 fusion protein described herein.

The invention also provides methods for inhibiting tumor growth in a subject comprising administering to the subject an effective amount of a p53 protein or p53 fusion protein described herein.

The invention also provides methods for delaying progression of cancer in a subject comprising administering to the subject an effective amount of a p53 protein or p53 fusion protein described herein.

The invention also provides methods for inhibiting angiogenesis having cancer comprising administering to the subject an effective amount of a p53 protein or p53 fusion protein described herein.

The invention also provides methods for delaying development of metastasis in a subject comprising administering to the subject an effective amount of a p53 protein or p53 fusion protein described herein.

In some variations, the subject has been diagnosed with cancer. In some variations, the cancers to be treated are p53-deficient, such as certain breast, ovarian, prostate, and head and neck cancers. For example, the p53 protein in the cancer cell is mutated and has reduced or diminished biological activity as compared to wild type p53.

Various formulations of p53 proteins or p53 fusion proteins may be used for administration. In some variations, a p53 protein or p53 fusion protein may be administered neat. In some variations, a p53 protein or p53 fusion protein and a pharmaceutically acceptable excipient are administered, and may be in various formulations. Pharmaceutically acceptable excipients are known in the art, and are relatively inert substances that facilitate administration of a pharmacologically effective substance. For example, an excipient can give form or consistency, or act as a diluent. Suitable excipients include but are not limited to stabilizing agents, wetting and emulsifying agents, salts for varying osmolarity, encapsulating agents, buffers, and skin penetration enhancers. Excipients as well as formulations for parenteral and nonparenteral drug delivery are set forth in Remington, The Science and Practice of Pharmacy 20th Ed. Mack Publishing (2000).

Generally, these agents are formulated for administration by injection (e.g., intraperitoneally, intravenously, subcutaneously, intratumorally, intramuscularly, etc.), although other forms of administration (e.g., oral, mucosal, etc) can be also used. Administration can be systemic, e.g., intravenous and intraperitoneal, or localized. A p53 protein or p53 fusion protein may be administered via site-specific or targeted local delivery techniques. Examples of site-specific or targeted local delivery techniques include various implantable depot sources of the protein or local delivery catheters, such as infusion catheters, an indwelling catheter, or a needle catheter, synthetic grafts, adventitial wraps, shunts and stents or other implantable devices, site specific carriers, direct injection, or direct application. Accordingly, the fusion proteins are preferably combined with pharmaceutically acceptable vehicles such as saline, Ringer's solution, dextrose solution, and the like.

The particular dosage regimen, i.e., dose, timing and repetition, will depend on the particular individual and that individual's medical history. Generally, any of the following doses may be used: a dose of at least about 50 mg/kg body weight; at least about 20 mg/kg body weight; at least about 10 mg/kg body weight; at least about 5 mg/kg body weight; at least about 3 mg/kg body weight; at least about 1 mg/kg body weight; at least about 750 μg/kg body weight; at least about 500 μg/kg body weight; at least about 250 ug/kg body weight; at least about 100 μg/kg body weight; at least about 50 μg/kg body weight; at least about 10 ug/kg body weight; at least about 1 μg/kg body weight, or more, is administered. Empirical considerations, such as the half-life, generally will contribute to determination of the dosage.

In some individuals, more than one dose may be required. Frequency of administration may be determined and adjusted over the course of therapy, and is based on reducing the number of cancerous cells, maintaining the reduction of cancerous cells, reducing the growth and/or proliferation of cancerous cells, or delaying the development of metastasis. The presence of cancerous cells can be identified by any number of methods known to one of skill in the art or discussed herein (e.g., detection by immunohistochemistry or flow cytometry of biopsies or biological samples). In some cases, sustained continuous release formulations of a p53 fusion protein may be appropriate. Various formulations and devices for achieving sustained release are known in the art.

In one embodiment, dosages may be determined empirically in individuals who have been given one or more administration(s). Individuals are given incremental dosages of p53 proteins or p53 fusion proteins. To assess efficacy of a p53 protein or p53 fusion protein, markers of the specific cancer disease state can be monitored. These markers include: direct measurements of tumor size via palpation or visual observation; indirect measurement of tumor size by x-ray or other imaging techniques; an improvement as assessed by direct tumor biopsy and microscopic examination of the tumor sample; the measurement of an indirect tumor marker (e.g., PSA for prostate cancer), a decrease in pain or paralysis; improved speech, vision, breathing or other disability associated with the tumor; increased appetite; or an increase in quality of life as measured by accepted tests or prolongation of survival. It will be apparent to one of skill in the art that the dosage will vary depending on the individual, the type of cancer, the stage of cancer, whether the cancer has begun to metastasize to other location in the individual, and the past and concurrent treatments being used.

Other formulations include suitable delivery forms known in the art including, but not limited to, carriers such as liposomes. See, for example, Mahato et al. (1997) Pharm. Res. 14:853-859. Liposomal preparations include, but are not limited to, cytofectins, multilamellar vesicles and unilamellar vesicles.

Compositions of the invention may be used in conjunction with other cancer therapies, for example, radiation therapies or chemotherapeutic agents (e.g., cisplatin, carboplatin, and oxaliplatin). Compositions of the invention may be administered in conjunction with other cancer therapeutic agents, such as Rituxan® and Herceptin®.

Assessment of disease is performed using standard methods in the arts, such as imaging methods and monitoring appropriate marker(s).

The invention also provides kits for therapeutic use. The kits of the invention comprises one or more p53 proteins or p53 fusion proteins. Kits of the invention may also include one or more containers comprising one or more purified p53 proteins or p53 fusion proteins and instructions for use in accordance with any of the methods of the invention described herein. Generally, these instructions comprise a description of administration of a p53 protein or p53 fusion protein to treat cancer according to any of the methods described herein. The kit may further comprise a description of selecting an individual suitable for treatment based on identifying whether that individual has cancer and the stage of the cancer.

The instructions relating to the use of the p53 protein or p53 fusion protein generally include information as to dosage, dosing schedule, and route of administration for the intended treatment. The containers may be unit doses, bulk packages (e.g., multi-dose packages) or sub-unit doses. Instructions supplied in the kits of the invention are typically written instructions on a label or package insert (e.g., a paper sheet included in the kit), but machine-readable instructions (e.g., instructions carried on a magnetic or optical storage disk) are also acceptable. The label or package insert indicates that the composition is used for treating cancer. Instructions may be provided for practicing any of the methods described herein.

The kits of this invention are in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump. A kit may have a sterile access port (for example the container may be an intravenous solution bag or a vial having a stopper pierceable by a hypodermic injection needle). The container may also have a sterile access port (for example the container may be an intravenous solution bag or a vial having a stopper pierceable by a hypodermic injection needle). At least one active agent in the composition is p53 fusion protein. The container may further comprise a second pharmaceutically active agent (such as one or more other cancer therapeutic agents).

Kits may optionally provide additional components such as buffers and interpretive information. Normally, the kit comprises a container and a label or package insert(s) on or associated with the container.

In some variations, the invention provides articles of manufacture comprising contents of the kits described above. In some embodiments, the kits comprise a p53 protein or a p53 fusion protein with information indicating use to treat cancer (e.g., lung and breast cancer). In some variations, the kits comprise a p53 protein or p53 fusion protein, and another anti-cancer agent with information indicating use to treat cancer in conjunction with each other.

The following Example is provided to illustrate but not limit the invention.

### Example 1

### Production of Tetrameric p53 Fusion Proteins in E. coli and Characterization of the p53 Fusion Proteins Generated

A p53-based protein therapeutic candidate may be wild-type to reduce immunogenicity, tetrameric to be efficient, and may be able to enter the targeted cancer cells. To date, attempts to produce wild-type, full-length, tetrameric p53 that is stable in the absence of chemical crosslinking have been unsuccessful. There are several reasons why isolation of p53 as a stable tetramer is important for a protein therapeutic application. First, the active form of p53 is a tetramer, p53 monomers bind DNA in a cooperative manner and the affinity for DNA is increased up to 100 fold by tetramerization (McLure, K G and Lee, P W 1998 EMBO J. 17:3342-3350). Second, the tetramerization domain is important for protein-protein interactions and tetramerization may regulate binding of some proteins to p53. The tetrameric structure may also allow for binding of multiple protein partners at the same time (Nie, Y et al. 2000 Mol Cell Biol 20:741-748; Liu, X et al. 1993 Mol Cell Biol 13:3291-3300; Marston, N J et al. 1995 Oncogene 10:1709-1715). Third, some post-translational modifications, for example phosphorylation and ubiquitination, require p53 to be oligomerized (Shieh, S Y et al. 2000 Genes Dev 14:289-300; Chene P. 2000 Biotechniques 28:240-242; Kubbutat, M H et al. 1998 Mol Cell Biol 18:5690-5698; Maki, C G 1999 J Biol Chem 274:16531-16535). Fourth, the nuclear export signal (NES) is located in the tetramerization domain (Stommel, J M et al. 1999 EMBO J. 18:1660-1672). It is exposed when p53 is monomeric but buried in the tetramer, allowing p53 to remain in the nucleus. Finally, and perhaps most importantly, tetrameric p53 may not heterotetramerize with endogenous mutant p53, an interaction that may decrease the effectiveness of a p53 therapeutic. In this example, the production of a wild-type tetrameric p53 that binds DNA and induces apoptosis when introduced into p53-deficient cancer cell lines is described. In addition, a stable tetrameric gonadotropin releasing hormone-p53 fusion protein (GnRH-p53) was produced and the protein as a potential novel protein therapeutic for the treatment of cancer was characterized.

Materials and Methods

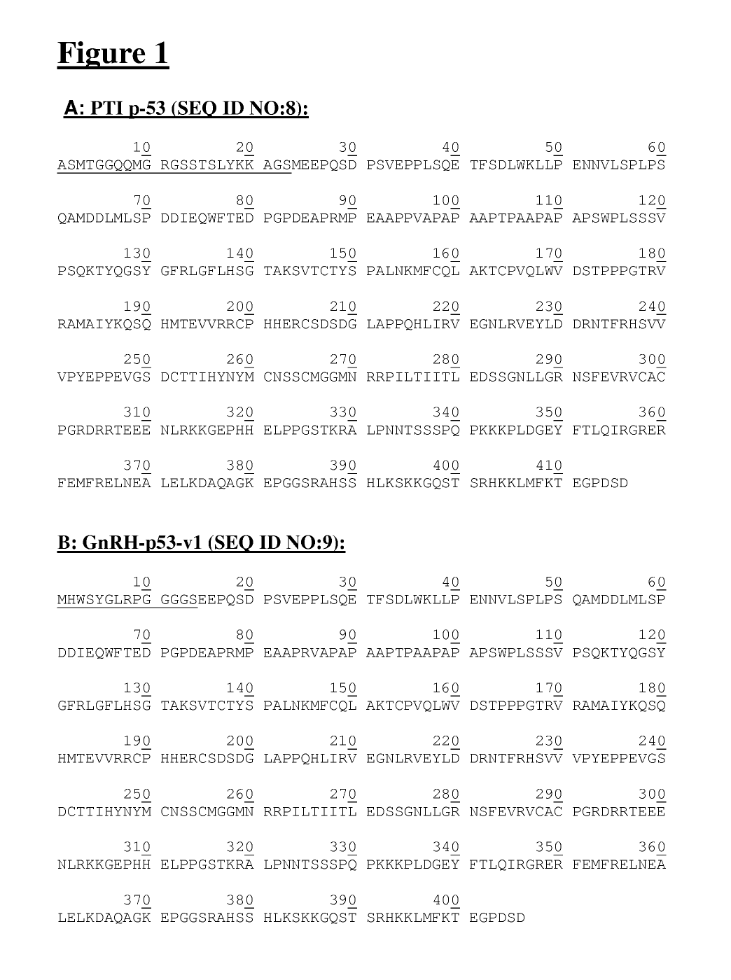

Construction of WT p53 and p53 fusion protein constructs. Wild-type p53 (accession number NM_{—}000546) was used as a PCR template for cloning of both wild type and the fusion protein, GnRH-p53. Oligonucleotides were designed to introduce convenient restriction endonuclease sites, and for the fusion protein, the GnRH sequence at the 5′-end. In addition, a polyglycine linker was installed between the coding region of GnRH and the coding region of p53 in order to minimize possible structural interference between these two domains. The following PCR primers were used: N2,5′-CAT ATG CAT TGG AGC TAT GGT CTG CGT CCG GGC GGC GGT AGC GAG-3′ (SEQ ID NO:14); N1,5′-CGT CCG GGC GGC GGT AGC GAG GAG CCG CAG TCA GAT C-3′ (SEQ ID NO:15); and C1,5′-GGT ACC TCA GTC TGA GTC AGG CCC TTC-3′ (SEQ ID NO:16). The PCR product was cloned into the NdeI/KpnI site of a modified pET11a vector for expression and the resulting expression plasmids are called pET11a-p53 and pET11a-GnRH-p53, respectively. The protein sequences of the expression constructs are shown in FIGS. 1A and 1B.

Expression and Purification. Escherichia coli BL21 (DE3) cells were transformed with pET11a-p53 and pET11a-GnRH-p53, and the expression and inclusion body purification were performed essentially as described (Kim, Y T et al. 2002 Eur J Biochem 269:5668-5677; Lin, X L et al. 1994 Methods Enzymol 241:195-224; Lin, X U.S. Pat. No. 6,583,268). Briefly, inclusion bodies were solubilized in buffer containing 8 M urea, 0.1 M Tris, 1 mM glycine, 10 mM B-ME, 10 mM DTT, 1 mM reduced glutathione, pH 10.5, at a final concentration of 2 mg/ml. Inclusion bodies were diluted 20-fold into refolding buffer containing 20 mM Tris, 0.2 M Arginine, 0.1 mM ZnCl₂, followed by slowly adjusting the pH to 8.0. Refolded proteins were concentrated using tangential flow filtration and purified using size exclusion chromatography (SEC) with a Sephacryl S-300 column (XK 50×850-mm, Amersham, Piscataway, N.J.) in refolding buffer with 0.1 M NaCl to separate soluble aggregates from refolded protein. SDS-PAGE was performed with a NuPAGE 4-12% Bis-Tris gel (Invitrogen) in MES buffer. Gels were stained with Coomassie Blue and destained in water.

Physical Characterization of Fusion Proteins: SDS-PAGE and Dynamic Light Scattering. The physical properties of the refolded p53 and GnRH-p53 were analyzed using SDS-PAGE and dynamic light scattering (DLS), and the resulting data were used as initial criteria for proper refolding of the proteins into stable tetramers. DLS was performed on purified samples (1-2 mg/ml) using Brookhaven Instruments Corporation 90 Plus particle size analyzer. Experiment preformed with a BIC BI-MAS Multi Angle Sizing Option on the Zeta PALS particle size analyzer. Data was analyzed using the DLSW research software package. DLS correlation function was fitted using a continuous distribution curve fit. Average effective diameter is reported.

In vitro DNA binding assay. The TransAm™ p53 DNA-binding kit (ActiveMotif, Carlsbad, Calif.) was used according to manufacturer's instructions to assess p53 DNA binding activity. A dose response curve of each purified p53 and GnRH-p53 was established, and the specific activity of each protein preparation was determined. Specificity of DNA binding was determined using competitive wild-type and mutant oligonucleotides in the DNA binding assay. Activity was compared to commercially available p53 (ActiveMotif, catalog number 31103). Experiments were carried out with samples in triplicate and the standard error was determined. Each experiment was performed at least three times, and student's t-test was used to evaluate the statistical significance of the data.

Cell Culture and Treatments. DU145, MDA-MB-231 and OVCAR3 cells were obtained from the American Type Culture Collection (ATCC) and were maintained in RPMI supplemented with 10% FBS and Pen/Strep. For experimental treatments, cells were plated at a density of 60,000 cells per ml in 96-well dishes (100 μl), 8-chamber slides (200 μl), or 6 cm dishes (5 ml) 24 hr before treatments. For GnRH-treated cells, cells were treated with indicated concentration of recombinant human GnRH (Axxora, San Diego, Calif.) for 72 hr.

Cell Proliferation Assay. The ability of the fusion proteins to induce growth arrest was measured by an MTS proliferation assay using the CellTiter 96 Aqueous One kit (Promega) according to manufacturer's instructions. Cells were treated with GnRH-p53, GnRH or PTI-p53 for 72 hr. Plates were read using a Spectramax plate reader from Molecular Devices (Sunnyvale, Calif.). Experiments were carried out in triplicate and the standard error was determined. Each experiment was performed three times, and the statistical significance of the data was evaluated using student's t-test. To determine the IC₅₀, data were plotted as % control v. log concentration and the IC₅₀ was determined to be the value where 50% of cells remained alive.

Nucleosome ELISA. Nucleosome ELISAs were performed using a Nucleosome ELISA kit (CalBiochem) according to manufacturer's protocol. Cells in 5-cm dishes were treated with GnRH-p53 or PTI-p53 (20 μg/ml) for 48 hr. Cells were then harvested and nuclear lysates were prepared. A 1:5 dilution of lysate was used and data was normalized for nuclear lysate total protein concentration.

TUNEL Staining. The ability to induce apoptosis was analyzed using TUNEL staining with the Frag-E1™ kit (EMD Biosciences, San Diego, Calif.) according to manufacturer's instructions. Cells were treated with the appropriate protein for 48 hr. Cells were treated overnight with 100 μM nutlin (Calbiochem, San Diego, Calif.), an agonist of the p53-mdm2 interaction that regulates ubiquitination and degradation of p53 thereby stabilizing the protein. p53 was activated to induce apoptosis with 50 μg/ml methylmethanesulfonate (MMS), which has been shown to induce p53-specific pro-apoptotic gene expression, for 2-3 hr prior to TUNEL staining. Experiments were carried out with samples in triplicate and the standard error was determined. Each experiment was performed three times, and student's t-test was used to evaluate the statistical significance of our data. Images were captured with a TE300 inverted fluorescent microscope using Simple PCI software.

Immunocytochemistry. After plating for 24 hr, cells were treated with GnRH-p53 or control samples for 10, 60 and 120 min at 37° C. Treatment was terminated by removal of media and washing 3× with ice cold PBS. The cells were fixed in 4% PFA for 20 min at 4° C., washed 1× in cold PBS and were stored in cold PBS until the immunostaining procedure was started (3-5 h). Cells were then permeabilized for 1 hr in 5% human serum, 5% donkey serum, 0.1% TX-100, 0.01% saponin, and 1% milk in PBS. BP53-12 mouse monoclonal p53 antibodies (Genetex) were diluted 1:200 in PBS/0.5% milk/0.01% saponin/2% serum and were used to stain the permeabilized cells overnight. Cells were rinsed 3×15 min with PBS, stained with a secondary antibody (Molecular Probes Alexa 555 Donkey anti-mouse IgG1:500) for 1 hr. The stained cells were then washed 3×20 min in PBS followed by incubation with TOTO3 nuclear stain (Molecular probes). The cells were visualized on a Nikon Eclipse E800 fluorescent microscope with 40× objective and images were captured with ACT-1 software. Confocal Z-stack images (100×) were acquired on a Zeiss LSM510 equipped with Argon, 543 HeNe and 633 HeNe lasers and LSM510 (3.5 SP1.1) software. Stacks were analyzed and colocalization statistics were generated using Bitplane Imaris Suite (4.5.2).

### Results

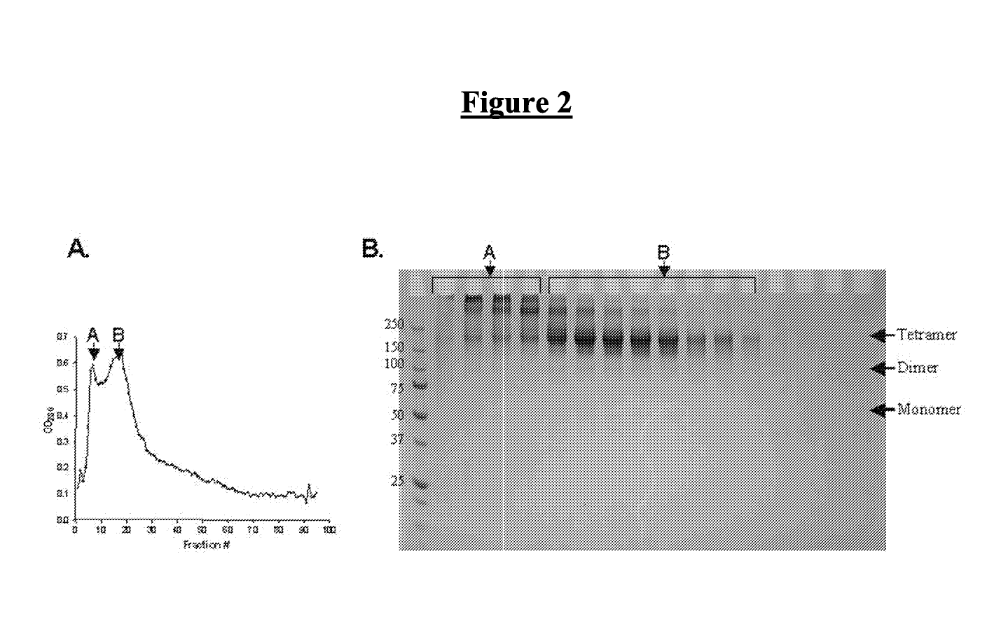

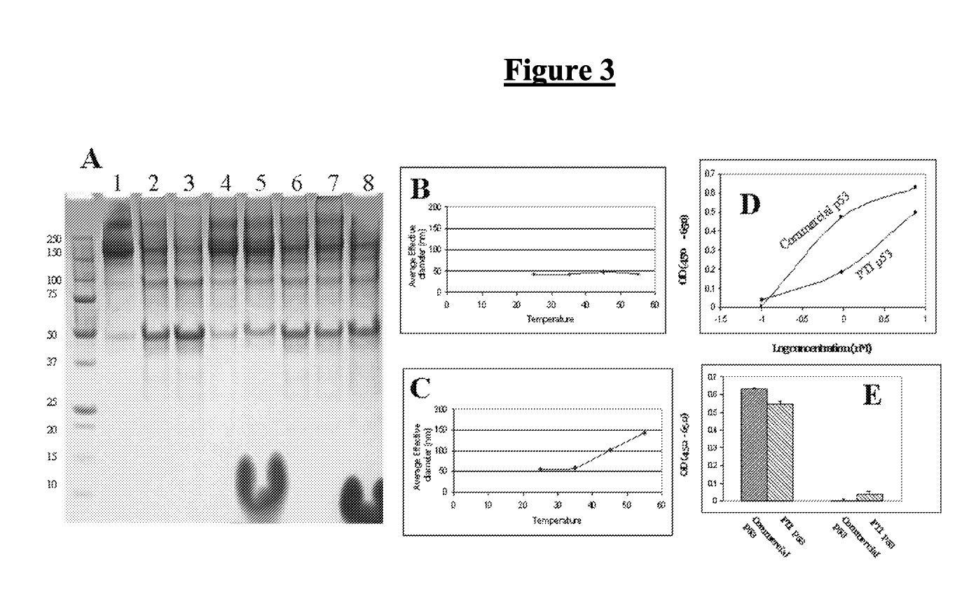

Stability of p53 tetramers. In order to test the possibility of developing p53 as a cancer therapeutic, wild type full length p53 protein was initially expressed in E. coli. In the expression construct, an additional N-terminal peptide was designed to facilitate high level expression in E. coli and inclusion body formation (FIG. 1A). The expressed p53 formed insoluble inclusion bodies which were subsequently purified and tested for different refolding conditions. Size exclusion chromatography (SEC) in native refolding buffer from one of the refolding conditions showed a peak in the tetrameric position (Peak B, FIG. 2A). Surprisingly, the tetramer is still stable when dissolved in a 0.1% SDS sample buffer and run on a SDS-PAGE (FIG. 2B). FIG. 3A (lane 1) shows that following an extended incubation at 37° C., a small amount of the tetramer dissociates into monomers, dimers, trimers, and some polymerized into multimers. High concentrations of urea disrupted the tetramer (lane 2). Furthermore, EDTA enhanced the disruption favoring formation of monomer (lane 3). On the other hand, both a negatively charged detergent, 0.25% N-Lauroylsarcosine, and a neutral detergent, 2.5% Tween 20, showed a protective or stabilizing effect for the p53 tetramer (lanes 4, 5). This “protection” effect is not as apparent when using three other detergents, TMAO, Zwittergent, and Nonidet P40 (lanes 6, 7, 8).

DLS was performed in order to confirm the presence of tetramer in solution at a low concentration of p53 (0.25 mg/ml, 4.7 μM) and to determine stability of the tetramer under stressed conditions. The effective diameter of monomeric p53 is approximately 9 nm (31). The DLS experiments demonstrated that the effective diameter of our p53 is approximately 45 nm (FIG. 3B). This roughly corresponds to the diameter of a tetrameric particle. The p53 tetramer has an increased Stokes' radius when compared to globular proteins of known molecular weight (Friedman, P N et al. 1993 Proc Natl Acad Sci USA 90:3319-3323). Upon heating to 55° C. in 5° C. increments, the protein did not undergo any increase in particle size that would correspond to unfolding or aggregation of unfolded protein. Therefore, tetrameric p53 is stable up to 55° C. under the experimental conditions. A different batch of p53 (FIG. 3C) was prepared under refolding conditions that favored the formation of soluble, misfolded p53 (judged by SDS-PAGE and DNA binding activity). This misfolded p53 has a higher polydispersity value (data not shown) and underwent heat-induced aggregate formation at 40° C. The sample in FIG. 3C shows a slightly larger effective diameter than the properly folded sample in FIG. 3B, with an average diameter of 53 nm compared to 45 nm. The difference in particle size observed may be explained by the fact that oxidized p53 is 18% larger than reduced p53, a value that corresponds with our experimental DLS data for FIG. 3C. SEC experiments demonstrate that mild oxidation of p53 results in the formation of monomers and high molecular weight species (Sun, X Z et al. 2003 Antioxid Redox Signal 5:655-665), consistent with our results shown in FIG. 3C.

DNA binding assay. A DNA binding assay was used to access the in vitro activity of PTI-p53. Native p53 oligomerizes in a concentration dependent manner, and DNA binding involves cooperativity between the subunits of the active tetramer. FIGS. 3D and 3E show several interesting observations. First, the degree to which binding is dependent on concentration of p53 is lower for PTI-p53 compared to commercially available p53. This is presumably due to the fact that tetramerization is concentration dependent, and PTI-p53 is already tetrameric at these concentrations. This characteristic may be important for a p53 therapeutic because tetrameric p53 may be more effective at a lower dose than monomeric p53. Second, at a relatively high concentration of p53 (7.5 nM, 390 μg/ml), PTI-p53 is 0.85 times as active as commercially available recombinant p53. However at low concentrations (0.1 nM, 0.005 μg/ml), PTI-p53 is 12.5 times as active as the commercially available p53 (FIG. 3E). Again, this is presumably due to the pre-oligomerized state of PTI-p53.

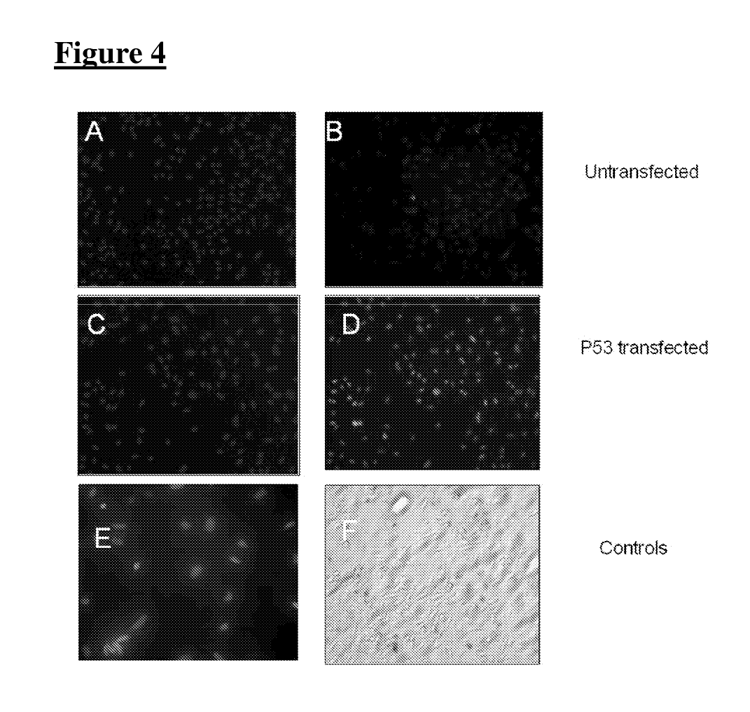

PTI-p53 inhibits cell proliferation and induced apoptosis in p53-deficient cell lines. Preliminary experiments were carried out to determine if PTI-p53 causes growth arrest or apoptosis when delivered into p53-deficient cells. PTI-p53 was transfected into p53-deficient PC3 prostate cancer cells or SaOS2 osteosarcoma cells using the protein transfection reagent Chariot™. This reagent relies on non-covalent binding of the Chariot reagent amphipathic peptide carrier to the protein of interest (Morris, M C et al. 2001 Nat Biotechnol 19:1173-1176). The efficiency of transfection was about 30% as judged by a parallel transfection of B-galactosidase followed by X-gal staining (FIG. 4F). Cells were treated with nutlin to stabilize p53 and methylmethanesulfonate to induce a p53-dependent genotoxic response (Amundson, S A et al. 2005 Oncogene 24:4572-4579; Vassilev, L T et al. 2004 Science 303:844-848). Several methods were used to assay cellular p53 activity including an MTS proliferation assay, a nucleosome formation assay and TUNEL staining. Results showed that PTI-p53 induced apoptosis when transfected into p53 deficient cell lines (FIG. 4D).

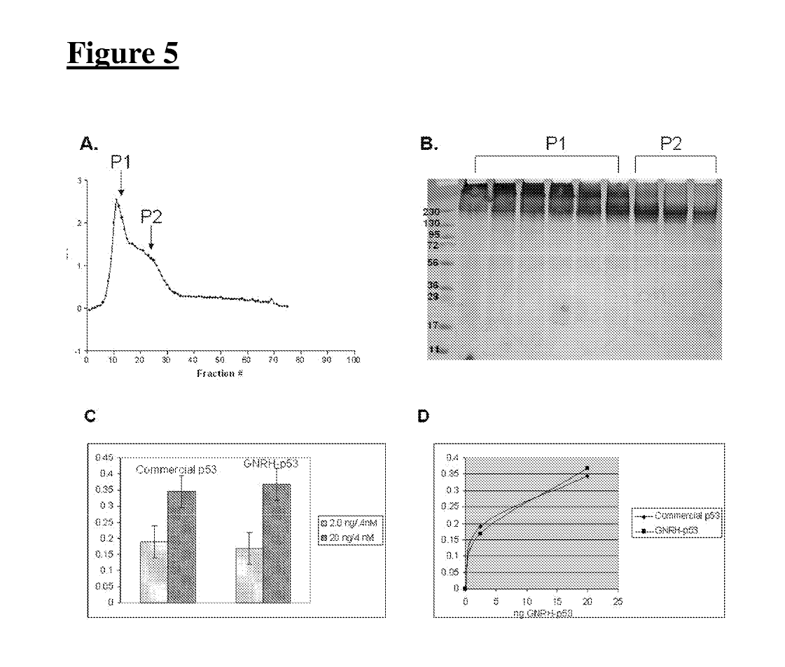

Expression, refolding, and in vitro functional test of GnRH-p53. In order to develop p53 protein as a cancer therapeutic, the protein needs to be delivered intracellularly. One of the delivery strategies is targeting specific cancer cells though receptor mediated endocytosis. Accordingly we designed a GnRH-p53 fusion protein to target GnRH-receptor (GnRH-R) positive cancer cells. The bacterial expression and refolding of the GnRH-p53 fusion protein was comparable to that of wild-type p53 (FIGS. 5A and 5B). In addition, GnRH-p53 shows a similar DNA binding profile with commercial p53 (FIGS. 5C and 5D).

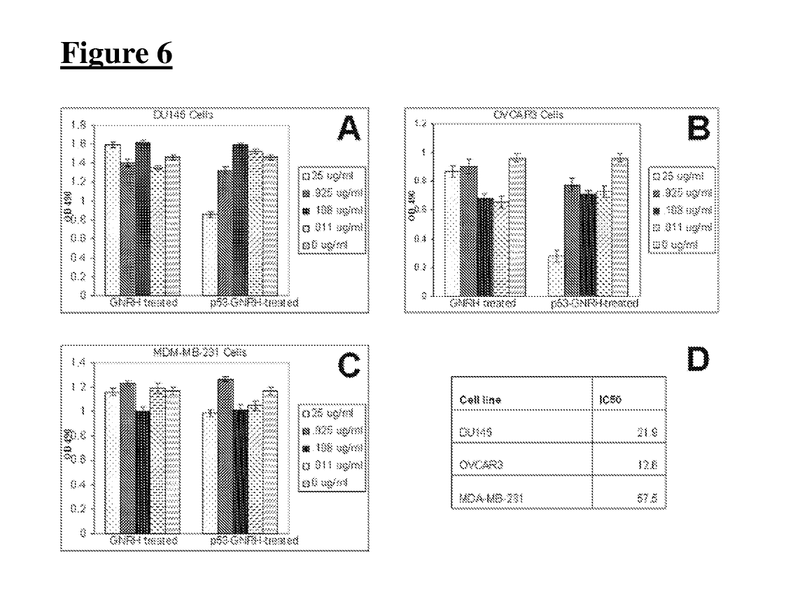

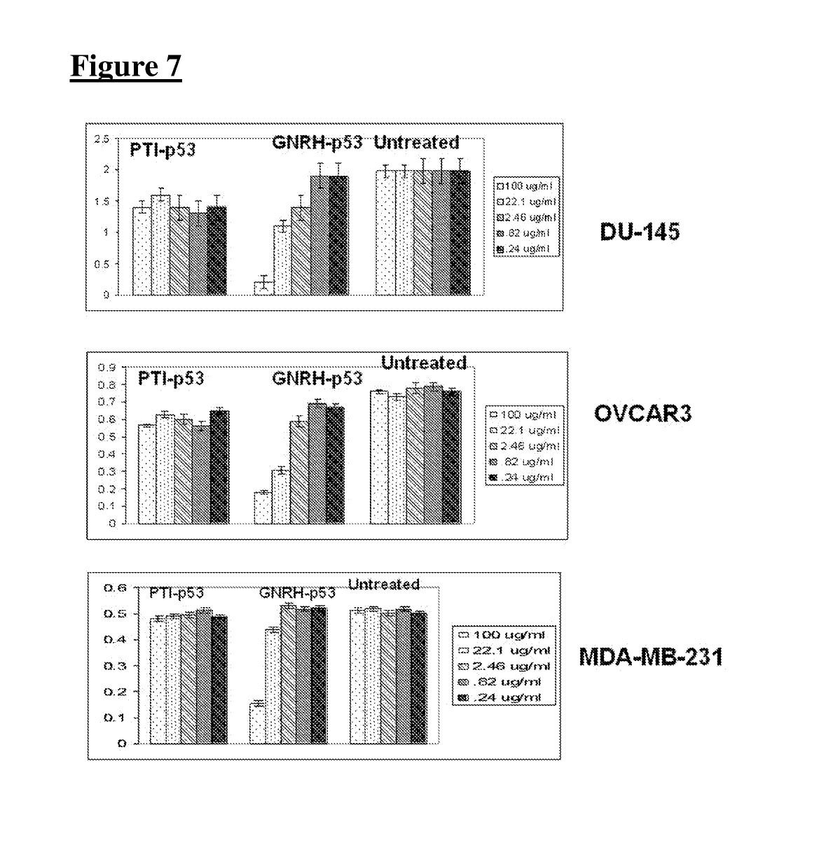

MTS proliferation assay. Three p53-deficient, GnRH-R expressing cancer cell lines were chosen to test the function of GnRH-p53 fusion protein. These cell lines are: DU145 (prostate cancer), OVCAR3 (ovarian cancer), and MDM-MB-231 (breast cancer). We first tested the effect of GnRH-p53 treatment on cell proliferation, which is shown in FIG. 6. After 72 hr of treatment with GnRH-p53, a 2 to 3-fold reduction in cellular proliferation was observed with the highest concentration of GnRH-p53 tested (25 μg/ml or 0.5 μM). No effect of GnRH treatment on proliferation was observed, even at more than 40 times molar concentrations (25 μg/ml or 22 μM), clearly indicating that the effect on proliferation of these cells lines is not due to GnRH, but to the GnRH-p53 fusion protein. In a separate experiment, we showed that wild type p53 had no effect on proliferation (FIG. 7). The IC₅₀ values for GnRH-p53 for each cell line are shown in FIG. 6D. There appears to be a difference in the sensitivity of different cell lines to treatment with GnRH-p53, which may reflect differences in GnRH-R expression or p53 sensitivity of different cell lines. FIG. 6 shows that among the three cell lines tested, MDA-MB-231 is least sensitive to the GnRH-p53 treatment.

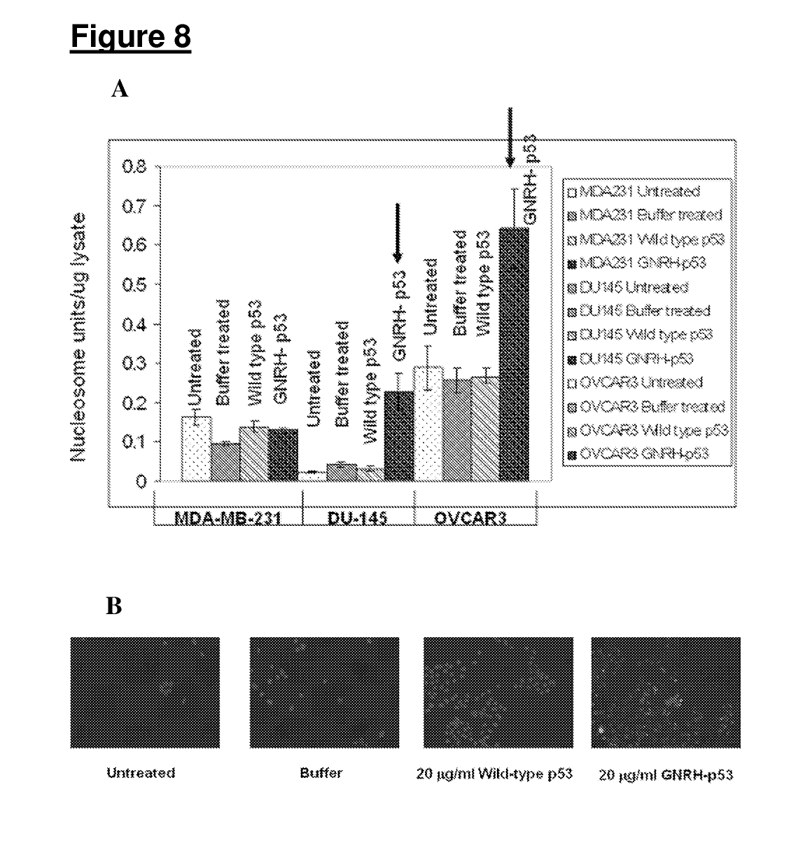

Apoptosis assay. In order to determine if the effect on proliferation was due to apoptosis induced by GnRH-p53, two apoptosis assays were performed, a nucleosome formation assay (early apoptotic event) and TUNEL staining (late apoptotic event). Results of the nucleosome formation ELISA assay are shown in FIG. 8A. In this figure, when cells were treated for 48 hr with GnRH-p53 (20 μg/ml), nucleosome formation was stimulated 3 and 10-fold in OVCAR3 and DU145 cells, respectively. Similar to the proliferation assay, the MDA-MB-231 cells did not shown apparent apoptosis in the concentrations of GnRH-p53 tested.

DNA fragmentation, a late apoptotic event, was detected most robustly in the OVCAR3 cells by TUNEL staining (FIG. 8B), but not in the DU145 or MDA-MB-231 cells with the concentration of GnRH-p53 tested. OVCAR3 cells were treated for 48 hr with GnRH-p53 (20 μg/ml), and a fluorescent TUNEL assay was performed. Treatment with GnRH-p53 (25 μg/ml) induced positive TUNEL staining in approximately 30% of the cells, but wild-type p53 (PTI-p53) had no effect (FIG. 8B).

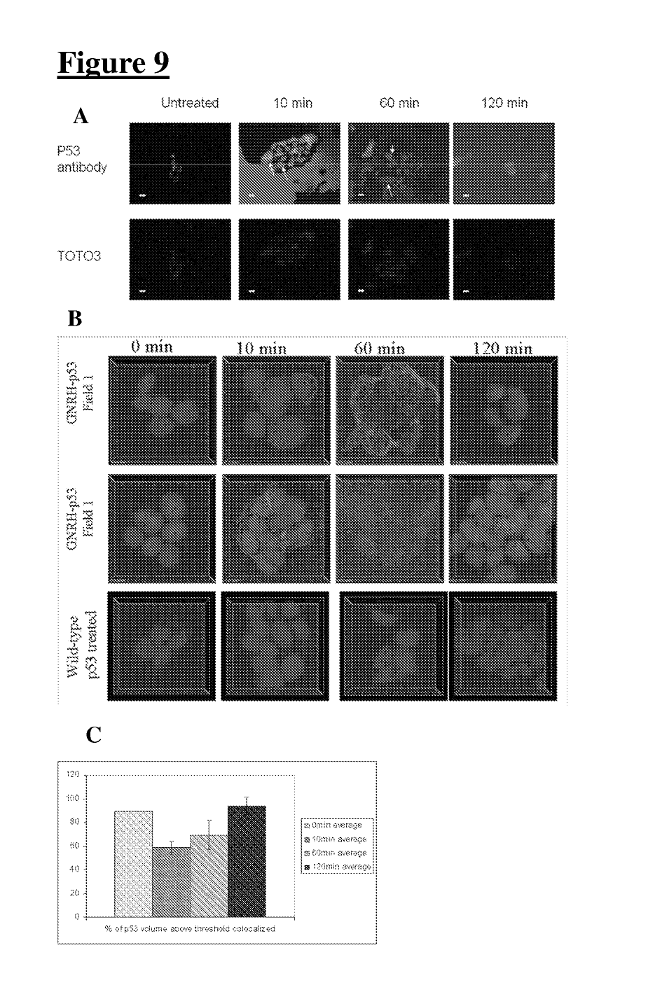

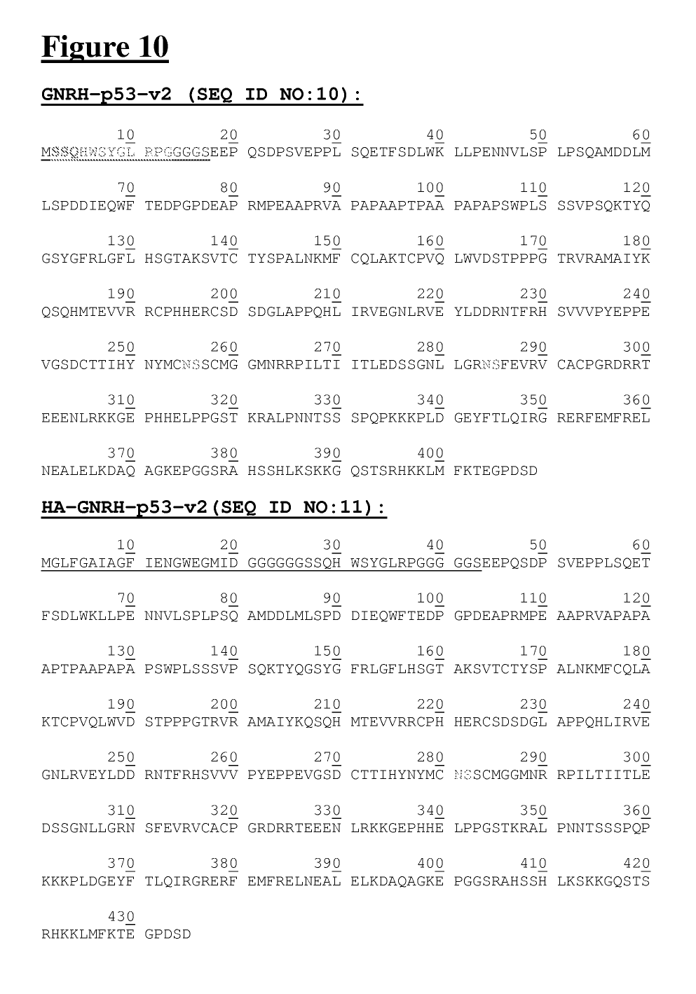

Immunocytochemistry. Internalization and translocation of GnRH-p53 was detected by immunocytochemistry. The p53 antibody used in this study detects both mutant and wild-type p53, therefore will recognize both endogenous mutant p53 and the GnRH-p53 fusion protein (FIGS. 9A and 9B). Cells were treated with GnRH-p53 (20 μg/ml) for 10, 60, or 120 min to capture receptor binding (10 min), internalization and nuclear translocation (60 and 120 min) of the GnRH-p53 fusion protein. Endogenous p53 is localized exclusively in the nucleus before treatment. After ten min of GnRH-p53 treatment, the fusion protein is localized to the plasma membrane and demonstrates punctuate staining in the cytoplasm (arrows, FIG. 9A). By 60 min the fusion protein is localized to the nuclear membrane and nucleus and cytoplasm as seen by punctuate staining in both (arrows, FIG. 9A). At 120 min, all p53 is localized to the nucleus exclusively. Results from confocal microscopy (FIG. 9B) are consistent with the results shown in FIG. 9A. FIG. 9C shows graphically the colocalization statistics from two individual fields (normalized for background staining, analyzed by volume) of GnRH-p53 with TOTO3 nuclear stain generated using Bitplane Imaris Suite (4.5.2). At 0 min, about 90% of p53 colocalized to nuclear, indicating a near-exclusive nuclear localization of the endogenous mutant p53. At 10 min, the colocalization decreased to about 60% in the GnRH-p53 samples, indicating the cell membrane localization of the exogenous GnRH-p53. The internalization of the GnRH-p53 is indicated at the 60 min point, which increased to about 70% colocalization. At 120 min, nearly all the GnRH-p53 was transported to the nuclear, evidenced by about 94% colocalization.

### SUMMARY

This example demonstrates the feasibility of using tetrameric p53 as a cancer therapeutic. First, full length, wild-type p53 was expressed and refolded into a stable tetramer. The refolded tetramer was stable in high concentrations of strong detergents such as SDS and N-Lauroylsarcosine, even in the presence of 8 M urea (FIG. 3a, lane 4), conditions that would disrupt the quaternary, tertiary, and even secondary structures of most proteins.

In order assist in the intracellular delivery of a p53 protein therapeutic, a targeting fusion protein was prepared containing a targeting peptide and full length p53. The peptide used in this example was GnRH, which has been shown to efficiently deliver small molecules, peptides and proteins into cells. GnRH is a secreted decapeptide that binds GnRH-R (King, J A and Millar, R P 1995 Cell Mol Neurobiol 15:5-23) which is expressed in the pituitary gland, ovary, placenta, breast and prostate tissues (Sealfon, S C et al. 1997 Endocr Rev 18:180-205). GnRH-R is also highly expressed in solid tumors and hormone-responsive cancer cell lines (Schally, A V et al. 2001 Front Neuroendocrinol 22:248-291). Therefore this strategy allows targeting of a p53 protein therapeutic to tumors in these hormone responsive tissue types.

Immunocytochemistry demonstrated that GnRH-p53 is rapidly bound to the GnRH-R on the cell surface (10 min), then internalized and translocated to the nucleus (60-120 min). After 120 min of treatment, elongation of the nuclei was seen which may indicate an immediate cellular effect of p53 on transcriptional regulation. The delayed effect on proliferation and cell death corresponds to the time required for initiation of p53-specific cell death and cell cycle gene expression. In addition, p53 requires extensive post-translational modifications in order for its activity to be regulated. The results shown here indicate that GnRH-p53 is functional to slow proliferation and induce apoptosis in p53-deficient cancer cell lines. A p53 fusion protein that is designed to cross the plasma membrane and be delivered into cells could be used to treat individuals with p53-deficient cancers in GnRH-responsive tissue types (ovarian, prostate, and breast) as a primary or secondary therapy.

It is known that efficient intracellular delivery of large proteins often requires more than just internalization. Some proteins that are internalized can remain trapped in the endosomes because escape from these vesicles is often inefficient (Wadia, J S et al. 2004 Nat Med 10:310-315). To overcome this potential limitation, strategies need to be devised for efficient “escape” of internalized proteins from endosomes. In order to do this, one of the strategies is to incorporate a peptide containing the N-terminal 20 amino acids from the influenza virus hemagglutinin protein (HA2) into the fusion protein. Influenza virus uses a mechanism of escape from endosomes that involves a pH-sensitive conformational change in the HA2 protein that destabilizes lipid membranes (2003 BioDrugs 17:216-222; Skehel, J J et al. 2001 Biochem Soc Trans 29:623-626). Fusion of a TAT domain to the 20 N-terminal amino acids of HA2 enhances the release of the fusion protein into the cytosol (Wadia, J S et al 2004 Nat Med 10:310-315).

Although the foregoing invention has been described in some detail by way of illustration and example for purposes of clarity of understanding, the descriptions and examples should not be construed as limiting the scope of the invention.

1. An isolated, biologically active p53 fusion protein in tetrameric form, wherein the p53 fusion protein comprises a p53 protein fused to one or more heterologous peptides.

2. The p53 fusion protein of claim 1, wherein the p53 protein is a human p53.

3. The p53 fusion protein of claim 1, wherein the p53 protein comprises the amino acid sequence selected from the group consisting of SEQ ID NO:7, residues 2-393 of SEQ ID NO:7, residues 3-393 of SEQ ID NO:7, residues 4-393 of SEQ ID NO:7, residues 5-393 of SEQ ID NO:7, and residues 6-393 of SEQ ID NO:7.

4. The p53 fusion protein of claim 1, wherein the p53 protein is fused to two or more heterologous peptides.

5. The p53 fusion protein of claim 1, wherein heterologous peptide is fuse to the N-terminus or the C-terminus of the p53 protein.

6. The p53 fusion protein of claim 5, wherein the heterologous peptide targets a cancer cell and internalizes the p53 fusion protein into the cancer cell.

7. The p53 fusion protein of claim 6, wherein the heterologous peptide is fused to the p53 protein through a linker peptide.

8. The p53 fusion protein of claim 1, wherein the heterologous peptide comprises the amino acid sequence selected from the group consisting of SEQ ID NOS:1-6.

9. The p53 fusion protein of claim 1, wherein the p53 fusion protein comprises the amino acid sequence selected from the group consisting of SEQ ID NOS:8-13.

10. The p53 fusion protein of claim 1, wherein the p53 fusion protein comprises the amino acid sequence selected from the group consisting of SEQ ID NOS:8-13, wherein the N-terminal amino acid methionine is removed.

11. The p53 fusion protein n of claim 1, wherein the p53 fusion protein is isolated and refolded from inclusion bodies from E. coli.

12. A composition comprising the p53 fusion protein of claim 1.

13. The composition of claim 12, further comprising a pharmaceutically acceptable excipient.

14. The composition of claim 12, wherein at least about 70% of the p53 fusion protein in the composition is in tetrameric form.

15. A p53 protein isolated and refolded from inclusion bodies from E. coli, wherein the p53 protein is in tetrameric form.

16. A composition comprising the p53 protein of claim 15.

17. The composition of claim 16, wherein at least about 70% of the p53 protein in the composition is in tetrameric form.

18. A method for producing a biologically active, tetrameric p53 protein or p53 fusion protein, comprising:
a) solubilizing a denatured p53 protein or p53 fusion protein with a solubilization buffer comprising a high concentration of chaotroph, a reducing agent, and having a pH of about 8.5 to about 12.0, to produce a solubilized p53 protein or p53 fusion protein solution;
b) diluting the solubilized p53 protein or p53 fusion protein solution with a refolding buffer by adding the solubilized p53 protein or p53 fusion protein solution into the refolding buffer to produce a diluted solubilized p53 protein or p53 fusion protein solution, wherein the refolding buffer comprises Tris, L-arginine, a detergent, a divalent cation ion, a chaotroph, or any combination thereof; and
c) reducing the pH of the diluted solubilized p53 protein or p53 fusion protein solution to a pH of about 7.5 to about 8.5, wherein said pH reducing is carried out over a period of at least about 20 hours, thereby producing a refolded, biologically active tetrameric p53 protein or p53 fusion protein.

19. The method of claim 18, wherein the solubilizing buffer comprises about 8 M urea, about 10 mM B-mercaptoethanol, about 10 mM dithiothreitol (DTT), and about 1 mM reduced glutathion (GSH) at pH about 10 to about 12.

20. The method of claim 18, further comprising adjusting the A280 of the solubilized p53 protein or p53 fusion protein solution to about 2.0 to about 10.0 before step (b).

21. The method of claim 20, wherein the A₂₈₀ of the solubilized p53 protein or p53 fusion protein solution is adjusted by diluting the solubilized p53 protein or p53 fusion protein solution in a buffer comprising about 8 M urea, about 10 mM β-mercaptoethanol, about 10 mM dithiothreitol (DTT), and about 1 mM reduced glutathion (GSH) at pH about 10 to about 12.

22. The method of claim 18, wherein the chaotroph is urea or guanidine hydrochloride.

23. The method of claim 18, wherein the refolding buffer comprises about 0.05 to about 1 M L-Arginine.

24. The method of claim 18, wherein the refolding buffer comprises about 0.1 M to about 2 M urea.

25. The method of claim 18, wherein the refolding buffer comprises about a detergent selected from the group consisting of Tween 20, Tween 80, N-Lauroylsarcosine, and sodium dodecyl sulfate.

26. The method of claim 19, wherein the refolding buffer comprises about 20 mM Tris, about 0.2 M L-arginine, about 0.1 mM ZnCl₂, pH about 10.5.

27. The method of claim 26, the solubilized p53 protein or p53 fusion protein solution is diluted about 20-fold with the refolding buffer in step b), and the pH is reduced to a pH of about 8.0 in step c).

28. The method of claim 18, wherein the denatured p53 protein or p53 fusion protein is from bacterial inclusion bodies.

29. The method of claim 18, further comprising purifying the refolded p53 protein or p53 fusion protein.

30. The method of claim 29, wherein the refolded p53 protein or p53 fusion protein is purified by one or more chromatography methods selected from the group consisting of size exclusion chromatography (SEC), ion exchange chromatography (IEC), hydrophobic interaction chromatography (HIC), and affinity chromatography.

31. The method of claim 18, wherein the p53 fusion protein comprises the amino acid sequence selected from the group consisting of SEQ ID NOS:8-13.

32. A purified tetrameric p53 protein or p53 fusion protein produced by the method of claim 29.

33. A method for treating cancer in an individual comprising administering to the individual in need thereof an effective amount of the p53 fusion protein of claim 1.

34. The method of claim 33, wherein the p53 fusion protein is administered in conjunction with one or more other cancer therapeutic agents.

35. The method of claim 33, wherein the p53 protein expressed in the cancer cells in the individual is mutated and has reduced or diminished biological activity as compared to a wild type p53.

36. A kit for treating cancer comprising the p53 fusion protein of claim 1.

37. The kit of claim 35, further comprising one or more other cancer therapeutic agents.


SEQUENCE LISTING (23 sequences)

SEQ ID NO: 1 - protein, 9 aa, Artificial Sequence
  Synthetic Construct
HWSYGLRPG

SEQ ID NO: 2 - protein, 12 aa, Artificial Sequence
  Synthetic Construct
SSQHWSYGLRPG

SEQ ID NO: 3 - protein, 11 aa, Artificial Sequence
  Synthetic Construct
RRRRRRRRRRR

SEQ ID NO: 4 - protein, 12 aa, Artificial Sequence
  Synthetic Construct
GRKKRRQRRRPP

SEQ ID NO: 5 - protein, 19 aa, Artificial Sequence
  Synthetic Construct
GLFGAIAGFIENGWEGMID

SEQ ID NO: 6 - protein, 25 aa, Artificial Sequence
  Synthetic Construct
GLFGAIAGFIENGWEGMIDGGGGGG

SEQ ID NO: 7 - protein, 393 aa, Homo sapien [sequence omitted]

SEQ ID NO: 8 - protein, 416 aa, Artificial Sequence
  Synthetic Construct
ASMTGGQQMGRGSSTSLYKKAGSMEEPQSDPSVEPPLSQETFSDLWKLLPENNVLSPLPSQAMDDLMLSPDDIEQWFTEDPGPDEAPRMPEAAPPVAPAPAAPTPAAPAPAPSWPLSSSVPSQKTYQGSYGFRLGFLHSGTAKSVTCTYSPALNKMFCQLAKTCPVQLWVDSTPPPGTRVRAMAIYKQSQHMTEVVRRCPHHERCSDSDGLAPPQHLIRVEGNLRVEYLDDRNTFRHSVVVPYEPPEVGSDCTTIHYNYMCNSSCMGGMNRRPILTIITLEDSSGNLLGRNSFEVRVCACPGRDRRTEEENLRKKGEPHHELPPGSTKRALPNNTSSSPQPKKKPLDGEYFTLQIRGRERFEMFRELNEALELKDAQAGKEPGGSRAHSSHLKSKKGQSTSRHKKLMFKTEGPDSD

SEQ ID NO: 9 - protein, 406 aa, Artificial Sequence
  Synthetic Construct
MHWSYGLRPGGGGSEEPQSDPSVEPPLSQETFSDLWKLLPENNVLSPLPSQAMDDLMLSPDDIEQWFTEDPGPDEAPRMPEAAPRVAPAPAAPTPAAPAPAPSWPLSSSVPSQKTYQGSYGFRLGFLHSGTAKSVTCTYSPALNKMFCQLAKTCPVQLWVDSTPPPGTRVRAMAIYKQSQHMTEVVRRCPHHERCSDSDGLAPPQHLIRVEGNLRVEYLDDRNTFRHSVVVPYEPPEVGSDCTTIHYNYMCNSSCMGGMNRRPILTIITLEDSSGNLLGRNSFEVRVCACPGRDRRTEEENLRKKGEPHHELPPGSTKRALPNNTSSSPQPKKKPLDGEYFTLQIRGRERFEMFRELNEALELKDAQAGKEPGGSRAHSSHLKSKKGQSTSRHKKLMFKTEGPDSD

SEQ ID NO: 10 - protein, 409 aa, Artificial Sequence
  Synthetic Construct
MSSQHWSYGLRPGGGGSEEPQSDPSVEPPLSQETFSDLWKLLPENNVLSPLPSQAMDDLMLSPDDIEQWFTEDPGPDEAPRMPEAAPRVAPAPAAPTPAAPAPAPSWPLSSSVPSQKTYQGSYGFRLGFLHSGTAKSVTCTYSPALNKMFCQLAKTCPVQLWVDSTPPPGTRVRAMAIYKQSQHMTEVVRRCPHHERCSDSDGLAPPQHLIRVEGNLRVEYLDDRNTFRHSVVVPYEPPEVGSDCTTIHYNYMCNSSCMGGMNRRPILTIITLEDSSGNLLGRNSFEVRVCACPGRDRRTEEENLRKKGEPHHELPPGSTKRALPNNTSSSPQPKKKPLDGEYFTLQIRGRERFEMFRELNEALELKDAQAGKEPGGSRAHSSHLKSKKGQSTSRHKKLMFKTEGPDSD

SEQ ID NO: 11 - protein, 435 aa, Artificial Sequence
  Synthetic Construct
MGLFGAIAGFIENGWEGMIDGGGGGGSSQHWSYGLRPGGGGGSEEPQSDPSVEPPLSQETFSDLWKLLPENNVLSPLPSQAMDDLMLSPDDIEQWFTEDPGPDEAPRMPEAAPRVAPAPAAPTPAAPAPAPSWPLSSSVPSQKTYQGSYGFRLGFLHSGTAKSVTCTYSPALNKMFCQLAKTCPVQLWVDSTPPPGTRVRAMAIYKQSQHMTEVVRRCPHHERCSDSDGLAPPQHLIRVEGNLRVEYLDDRNTFRHSVVVPYEPPEVGSDCTTIHYNYMCNSSCMGGMNRRPILTIITLEDSSGNLLGRNSFEVRVCACPGRDRRTEEENLRKKGEPHHELPPGSTKRALPNNTSSSPQPKKKPLDGEYFTLQIRGRERFEMFRELNEALELKDAQAGKEPGGSRAHSSHLKSKKGQSTSRHKKLMFKTEGPDSD

SEQ ID NO: 12 - protein, 410 aa, Artificial Sequence
  Synthetic Construct
MEEPQSDPSVEPPLSQETFSDLWKLLPENNVLSPLPSQAMDDLMLSPDDIEQWFTEDPGPDEAPRMPEAAPRVAPAPAAPTPAAPAPAPSWPLSSSVPSQKTYQGSYGFRLGFLHSGTAKSVTCTYSPALNKMFCQLAKTCPVQLWVDSTPPPGTRVRAMAIYKQSQHMTEVVRRCPHHERCSDSDGLAPPQHLIRVEGNLRVEYLDDRNTFRHSVVVPYEPPEVGSDCTTIHYNYMCNSSCMGGMNRRPILTIITLEDSSGNLLGRNSFEVRVCACPGRDRRTEEENLRKKGEPHHELPPGSTKRALPNNTSSSPQPKKKPLDGEYFTLQIRGRERFEMFRELNEALELKDAQAGKEPGGSRAHSSHLKSKKGQSTSRHKKLMFKTEGPDSDPGGGGSRRRRRRRRRRR

SEQ ID NO: 13 - protein, 411 aa, Artificial Sequence
  Synthetic Construct
MEEPQSDPSVEPPLSQETFSDLWKLLPENNVLSPLPSQAMDDLMLSPDDIEQWFTEDPGPDEAPRMPEAAPRVAPAPAAPTPAAPAPAPSWPLSSSVPSQKTYQGSYGFRLGFLHSGTAKSVTCTYSPALNKMFCQLAKTCPVQLWVDSTPPPGTRVRAMAIYKQSQHMTEVVRRCPHHERCSDSDGLAPPQHLIRVEGNLRVEYLDDRNTFRHSVVVPYEPPEVGSDCTTIHYNYMCNSSCMGGMNRRPILTIITLEDSSGNLLGRNSFEVRVCACPGRDRRTEEENLRKKGEPHHELPPGSTKRALPNNTSSSPQPKKKPLDGEYFTLQIRGRERFEMFRELNEALELKDAQAGKEPGGSRAHSSHLKSKKGQSTSRHKKLMFKTEGPDSDPGGGGSGRKKRRQRRRPP

SEQ ID NO: 14 - DNA, 45 bp, Artificial Sequence
  Synthetic Construct
catatgcattggagctatggtctgcgtccgggcggcggtagcgag

SEQ ID NO: 15 - DNA, 37 bp, Artificial Sequence
  Synthetic Construct
cgtccgggcggcggtagcgaggagccgcagtcagatc

SEQ ID NO: 16 - DNA, 27 bp, Artificial Sequence
  Synthetic Construct
ggtacctcagtctgagtcaggcccttc

SEQ ID NO: 17 - protein, 6 aa, Artificial Sequence
  Synthetic Construct
PGGGGS

SEQ ID NO: 18 - protein, 9 aa, Artificial Sequence
  Synthetic Construct
ESGGGGSPG

SEQ ID NO: 19 - protein, 9 aa, Artificial Sequence
  Synthetic Construct
SPGGGGSPG

SEQ ID NO: 20 - protein, 9 aa, Artificial Sequence
  Synthetic Construct
APGAGAGPG

SEQ ID NO: 21 - protein, 4 aa, Artificial Sequence
  Synthetic Construct
GGGS

SEQ ID NO: 22 - protein, 6 aa, Artificial Sequence
  Synthetic Construct
GGGGGG

SEQ ID NO: 23 - protein, 5 aa, Artificial Sequence
  Synthetic Construct
GGGGS